SEQUENCE LISTING (3 sequences)

SEQ ID NO: 1 - protein, 248 aa, Artificial Sequence
  Synthetic Construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLSKADYEKHKVYACEVTHQGLSSPVTKSFNRGEC

SEQ ID NO: 2 - protein, 224 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - protein, 357 aa, Artificial Sequence [sequence omitted]

(19) United States
(12) Patent Application Publication
(10) Pub. No.: US 2020/0109432 A1
(43) Pub. Date: Apr. 9, 2020

(54) Analysis of Disulfide Bonds

(71) Applicant: Momenta Pharmaceuticals, Inc., Cambridge, MA (US)

(72) Inventors: Brian Collins, Cambridge, MA (US); John Robblee, Cambridge, MA (US); Nathaniel Washburn, Cambridge, MA (US); Yan Yin, Cambridge, MA (US); Enrique Arevalo, Cambridge, MA (US)

(21) Appl. No.: 16/505,333

(22) Filed: Jul. 8, 2019

Related U.S. Application Data
(63) Continuation of application No. 14/668,764, filed on Mar. 25, 2015, now abandoned.
(60) Provisional application No. 61/970,701, filed on Mar. 26, 2014.

(51) Int. Cl. C12Q 1/37; C07K 16/00; C07K 14/705; C07K 16/24

(52) U.S. Cl. CPC C12Q 1/37; C07K 16/00; C07K 14/70521; A61K 38/00; C07K 2317/56; C07K 2317/40; C07K 2319/70; C07K 16/241

(57) ABSTRACT

The present disclosure relates to methods of evaluating, identifying, and/or producing (e.g., manufacturing) pharmaceutical products (e.g., protein therapeutics). In some instances, methods herein allow highly resolved evaluation of the disulfide bond profiles of protein therapeutics.

### CLAIM OF PRIORITY

This application is a continuation of U.S. patent application Ser. No. 14/668,764, filed on Mar. 25, 2015, which claims priority U.S. Patent Provisional Application Ser. No. 61/970,701, filed on Mar. 26, 2014, the entire contents of which are hereby incorporated by reference.

### BACKGROUND

Disulfide bonds in proteins are formed between thiol groups of cysteine residues and play a role in the folding and stability of proteins.

### SUMMARY OF THE INVENTION

The present disclosure provides methods of evaluating, identifying, and/or producing (e.g., manufacturing) pharmaceutical products (e.g., protein therapeutics) based on the detection of predefined disulfide bond profiles in the products.

Accordingly, in a first aspect the invention features a method of manufacturing a pharmaceutical product. The method includes: obtaining a sample of a batch of a test biologic; determining a disulfide bond profile for the sample; acquiring an assessment made by comparing said determined disulfide bond profile with a disulfide bond profile of a target protein (e.g., a specification including a disulfide bond profile of a target protein), wherein the target protein is a biologic approved under a primary approval pathway; and processing the batch of the test biologic into a pharmaceutical product if the assessment reveals the determined disulfide bond profile conforms with the disulfide bond profile of the target protein; thereby manufacturing a pharmaceutical product.

In some embodiments, the determining step comprises digesting the sample with one or more protease and/or glycosidase (also referred to in the art as glycoside hydrolase, herein referred to collectively as “glycosidase”) enzymes in a digestion buffer (e.g., a digestion buffer including trypsin, flavastacin, LysC, GluC, and/or PNGase F (also referred to N-Glycanase)). In certain embodiments, the determining step includes digesting the sample with not more than one protease enzyme in a digestion buffer (e.g., a digestion buffer including trypsin). In other embodiments, the determining step includes digesting the sample with at least two (e.g., two, three, four, five, six, seven, eight, nine, or ten) protease enzymes in a digestion buffer (e.g., a digestion buffer including trypsin and GluC). In some embodiments, the determining step includes digesting the sample with no more than ten (e.g., no more than two, no more than three, no more than four, no more than five, no more than six, no more than seven, no more than eight, or no more than nine) protease enzymes in a digestion buffer (e.g., a digestion buffer including trypsin and GluC). In some embodiments, the digestion buffer further includes a glycosidase enzyme (e.g., PNGase F).

In some embodiments, the test biologic is an antibody (e.g., a monoclonal antibody, such as an IgG antibody, for example an IgG1 antibody). In certain embodiments, the antibody has a light chain with an amino acid sequence with at least 95% (e.g., at least 98%, at least 99%, or 100%) identity to SEQ ID NO:1 and a heavy chain with an amino acid sequence with at least 95% (e.g., at least 98%, at least 99%, or 100%) identity to SEQ ID NO:2.

In other embodiments, the test biologic is a fusion protein (e.g., an Fc fusion protein). In certain embodiments, the fusion protein has an amino acid sequence having at least 95% (e.g., at least 98%, at least 99%, or 100%) identity to SEQ ID NO:3.

In another aspect, the invention features a method of manufacturing a pharmaceutical product comprising an antibody having a light chain with an amino acid sequence having 100% identity to SEQ ID NO:1 and a heavy chain with an amino acid sequence having 100% identity to SEQ ID NO:2. This method includes: obtaining a sample of a batch of a test biologic, wherein the test biologic is an antibody having a light chain with an amino acid sequence having 100% identity to SEQ ID NO:1 and a heavy chain with an amino acid sequence having 100% identity to SEQ ID NO:2, and wherein the test antibody is approved under a secondary approval pathway; determining a disulfide bond profile for the sample, wherein the determining includes digesting the sample with no more than one protease enzyme in a digestion buffer; acquiring an assessment made by comparing the determined disulfide bond profile with a disulfide bond profile of a target antibody (e.g., a specification including a disulfide bond profile of a target antibody) having a light chain with an amino acid sequence having 100% identity to SEQ ID NO:1 and a heavy chain with an amino acid sequence having 100% identity to SEQ ID NO:2, and wherein the target antibody is approved under a primary approval pathway; processing the batch of the test antibody into a pharmaceutical product including an antibody having a light chain with an amino acid sequence having 100% identity to SEQ ID NO:1 and a heavy chain with an amino acid sequence having 100% identity to SEQ ID NO:2 if the assessment reveals the disulfide bond profile of the sample conforms with the disulfide bond profile of the target antibody; thereby manufacturing a pharmaceutical product including an antibody having a light chain with an amino acid sequence having 100% identity to SEQ ID NO:1 and a heavy chain with an amino acid sequence having 100% identity to SEQ ID NO:2.

In another aspect, the invention features a method of manufacturing a pharmaceutical product including a fusion protein having an amino acid sequence having 100% identity to SEQ ID NO: 3. This method includes: obtaining a sample of a batch of test biologic, wherein the test biologic is a fusion protein having an amino acid sequence having 100% identity to SEQ ID NO: 3, and wherein the test protein is approved under a secondary approval pathway; determining a disulfide bond profile for the sample, wherein the determining comprises digesting the sample with no more than two protease enzymes in a digestion buffer; acquiring an assessment made by comparing the test protein disulfide bond profile with a disulfide bond profile of a target protein (e.g., a specification including a disulfide bond profile of a target protein) having an amino acid sequence having 100% identity to SEQ ID NO: 3, and wherein the target protein is approved under a primary approval pathway; processing the batch of the test protein into a pharmaceutical product including a fusion protein having an amino acid sequence having 100% identity to SEQ ID NO: 3 if the assessment reveals the disulfide bond profile of the sample conforms with the disulfide bond profile of the target protein; thereby manufacturing a pharmaceutical product including a fusion protein having an amino acid sequence having 100% identity to SEQ ID NO: 3.In another aspect, the invention features a method of manufacturing a pharmaceutical product including a fusion protein having an amino acid sequence having 100% identity to SEQ ID NO: 3. This method includes: obtaining a sample of a batch of test biologic, wherein the test biologic is a fusion protein having an amino acid sequence having 100% identity to SEQ ID NO: 3, and wherein the test protein is approved under a secondary approval pathway; determining a disulfide bond profile for the sample, wherein the determining comprises digesting the sample with no more than two protease enzymes in a digestion buffer; wherein said digestion buffer further includes a glycosidase enzyme; acquiring an assessment made by comparing the test protein disulfide bond profile with a disulfide bond profile of a target protein (e.g., a specification including a disulfide bond profile of a target protein) having an amino acid sequence having 100% identity to SEQ ID NO: 3, and wherein the target protein is approved under a primary approval pathway; processing the batch of the test protein into a pharmaceutical product including a fusion protein having an amino acid sequence having 100% identity to SEQ ID NO: 3 if the assessment reveals the disulfide bond profile of the sample conforms with the disulfide bond profile of the target protein; thereby manufacturing a pharmaceutical product including a fusion protein having an amino acid sequence having 100% identity to SEQ ID NO: 3.

In some embodiments of any of the foregoing methods, the digestion buffer includes trypsin, flavastacin, LysC, GluC, and/or PNGase F (e.g., trypsin and GluC or trypsin, GluC, and PNGase F).

In other embodiments of any of the foregoing methods, the digesting is performed in a controlled environment such that disulfide connectivity is essentially maintained (e.g., using pressure cycling technology).

In certain embodiments of any of the foregoing methods, the determining step further includes separating the digested sample to produce separated components of the sample.

In some embodiments of any of the foregoing methods, the determining step includes alkylating the sample with one or more alkylating agents under non-reducing conditions.

In other embodiments of any of the foregoing methods, the test protein disulfide bond profile is directly obtained by performing an analytical test on the test biologic preparation.

In certain embodiments of any of the foregoing methods, the disulfide bond profile is obtained using a method provided in Table 1.

In some embodiments of any of the foregoing methods, the processing step comprises combining the test biologic preparation with an excipient or buffer.

In other embodiments of any of the foregoing methods, the processing step comprises one or more of: formulating the test biologic preparation; processing the test biologic preparation into a drug product; combining the test biologic preparation with a second component; changing the concentration of the biologic in the preparation; lyophilizing the test biologic preparation; combining a first and second aliquot of the biologic to provide a third, larger, aliquot; dividing the test biologic preparation into smaller aliquots; disposing the test biologic preparation into a container; packaging the test biologic preparation; associating a container comprising the test biologic preparation with a label; and shipping or moving the test biologic to a different location.

In certain embodiments of any of the foregoing methods, the test biologic and/or the pharmaceutical product is not approved under a primary approval pathway. In some embodiments of any of the foregoing methods, the test biologic and/or the pharmaceutical product is not approved under Section 351(a) of the PHS Act. In other embodiments of any of the foregoing methods, the test biologic and/or the pharmaceutical product is approved under a secondary approval pathway. In certain embodiments of any of the foregoing methods, the test biologic and/or the pharmaceutical product is approved under Section 351(k) of the Public Health Service (PHS) Act.

In some embodiments of any of the foregoing methods, the disulfide bond profile of a target protein is for one, two, or more samples or batches. In other embodiments of any of the foregoing methods, the disulfide bond profile of a target protein is for an average of disulfide bond profiles for multiple batches.

In certain embodiments, the disulfide bond profile of a target protein is a specification for commercial release of a drug product under Section 351(k) of the Public Health Service Act.

In some instances, processing may include formulating, packaging (e.g., in a syringe or vial), labeling, or shipping at least a portion of the biologic preparation. In some instances, processing includes formulating, packaging (e.g., in a syringe or vial), and labeling at least a portion of the biologic as a protein therapeutic. Processing can include directing and/or contracting another party to process as described herein.

In some embodiments of any of the foregoing methods, the disulfide profile of the test protein and the disulfide profile of the target protein are determined with the same method.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

These and other aspects of the invention are described in more detail below and in the claims.

### BRIEF DESCRIPTION OF THE DRAWINGS

FIG. 1 depicts the amino acid sequences of SEQ ID NOs: 1, 2, and 3.

### DETAILED DESCRIPTION

The present disclosure provides that relationships between cysteines residues present within a biologic can be used as a signature (e.g., a product signature) of the biologic. As such, the disclosure provides compositions, activities, actions, and methods drawn to understanding the relationships between cysteines present in biologics. For example, the disclosure provides that information concerning the relationships between cysteines present in biologics, a so called disulfide bond profile, e.g., obtained from/for a sample of the biologic, can be used as a product signature to identify the biologic, e.g., as suitable for subsequent commercial activity.

### Definitions

As used herein, a biologic refers to naturally derived or recombinant products expressed in cells that are: (i) composed of amino acid sequences; and (ii) that include one or more disulfide-linked cysteine pairs. Exemplary biologics include antibodies, and antibody-like molecules (e.g., Fc fusion proteins) and antibody fragments (e.g., Fab fragments and Fc fragments).

A biologic preparation is a composition that includes at least one biologic. In some instances, the at least one biologic can include two or more isoforms. As used herein, the term isoform refers to any of two or more different forms of the same biologic that differ from one another with respect to one or more characteristic or feature, e.g., the presence or absence of a disulfide bond at any particular cysteine residue. The terms biologic and biologic preparation are used interchangeably with respect to the methods disclosed herein.

As used herein, a batch refers to a single production run, e.g., a commercial manufacturing run, of a biologic. Evaluation of different batches thus means evaluation of different production runs or batches. As used herein sample(s) refer to separately procured portions of a batch or batches. For example, evaluation of separate samples could mean evaluation of different commercially available containers or vials of the same batch or from different batches. A batch can include drug product or drug substance. As used herein, a primary approval process is an approval process which does not refer to a previously approved protein. In embodiments the primary approval process is one in which the applicant does not rely, for approval, on data, e.g., clinical data, from a previously approved product. Exemplary primary approval processes include, in the U.S, a Biologics License Application (BLA), or supplemental Biologics License Application (sBLA), a new drug application (NDA) under 505(b)(1) of the Federal Food and Cosmetic Act, and in Europe an approval in accordance with the provisions of Article 8(3) of the European Directive 2001/83/EC, or an analogous proceeding in other countries or jurisdictions.

As used herein, secondary approval process refers to an approval process which refers to clinical data for a previously approved product. In embodiments the secondary approval requires that the product being approved have structural or functional similarity to a previously approved product, e.g., a previously approved protein having the same primary amino acid sequence or a primary amino acid sequence that differs by no more than 1, 2, 3, 4, 5, or 10 residues or that has at least 98%, 99% or more (100%) sequence identity. In embodiments the secondary approval process is one in which the applicant relies, for approval, on clinical data from a previously approved product. Exemplary secondary approval processes include, in the U.S, an approval under 351(k) of the Public Health Service Act or under section 505(j) or 505(b)(2) of the Hatch Waxman Act and in Europe, an application in accordance with the provisions of Article 10, e.g., Article 10(4), of the European Directive 2001/83/EC, or an analogous proceeding in other countries or jurisdictions.

As used herein, evaluating, e.g., in the evaluation/evaluating, identifying, and/or producing aspects disclosed herein means reviewing, considering, determining, assessing, analyzing, measuring, and/or detecting the presence, absence, level, and/or ratio of a disulfide bond or disulfide bond profile in a sample. In some instances, evaluating can include performing a process that involves a physical change in a sample or another substance, e.g., a starting material. In some instances, evaluating a biologic includes measuring or detecting the presence, absence, level, or ratio of one or disulfide bonds, e.g., using methods disclosed herein.

Information, as used herein, can be qualitative, e.g., present, absent, intermediate, or quantitative, e.g., a numerical value such as a single number, or a range, for a parameter. In some instances, information can be a range or average (or other measure of central tendency), e.g., based on the values from any X samples or batches, e.g., wherein at least X of the samples or batches is being evaluated for commercial release, wherein X is equal to, at least, or no more than, 2, 3, 4, 5, 6, 7, 8, 9, 10, 11, 12, 13, 14, 15, 16, 17, 18, 19, 20, 21, 22, 23, or 24. In some instances, information can be, for example: a statistical function, e.g., an average, of a number of values; a function of another value, e.g., of the presence, distribution or amount of a second entity present in the sample, e.g., an internal standard; a statistical function, e.g., an average, of a number of values; a function of another value, e.g., of the presence, distribution or amount of a second entity present in the sample, e.g., an internal standard; a value, e.g., a qualitative value, e.g., present, absent, “below limit of detection,” “within normal limits,” or intermediate. In some instances, information can be a quantitative value, e.g., a numerical value such as a single number, a range of values, a “no less than x amount” value, a “no more than x amount” value. In some instances, information can be abundance. Abundance can be expressed in relative terms, e.g., abundance can be expressed in terms of the abundance of a structure in relation to another component in the preparation.

As used herein, acquire or acquiring (e.g., acquiring information) means obtaining possession of a physical entity, or a value, e.g., a numerical value, by directly acquiring or indirectly acquiring the physical entity or value. Directly acquiring means performing a process (e.g., performing an assay or test on a sample or analyzing a sample as that term is defined herein) to obtain the physical entity or value. Indirectly acquiring refers to receiving the physical entity or value from another party or source (e.g., a third party laboratory that directly acquired the physical entity or value).

### Disulfide Bond Profile

As used herein, the term disulfide bond profile refers to relationships between cysteine residues present in a biologic, which relationships serve as a signature of the biologic. As disclosed herein, relationships between cysteine residues, or information conveying those relationships, can be qualitative (e.g., relating to the presence, absence, location of disulfide linkages or bonds between cysteine residues) and/or quantitative (e.g., relating to occupancy and/or abundance of disulfide linkages or bonds between cysteine residues) and can relate to on-diagonal and/or off-diagonal disulfide linked cysteines and/or free cysteine residues.

As disclosed herein, a disulfide bond profile is a signature of a biologic, which signature can be used to identify a test biologic (e.g., a biologic approved under a secondary approval pathway) as a target biologic (e.g., a biologic approved under a primary approval pathway), and/or to signal further activity (e.g., processing, formulating, etc) related to the test biologic. In some instances, a disulfide bond profile is a specification for commercial release of a test biologic. In some instances, a disulfide bond profile is a specification for commercial release of a biologic approved under a secondary approval pathway. In some instances, a disulfide bond profile is a specification for commercial release of a biologic approved under Section 351(k) of the Public Health Service (PHS) Act. In some instances, a disulfide bond profile is a specification (e.g., a GMP standard, an FDA label or Physician's Insert) or quality criterion for a pharmaceutical preparation containing the target biologic.

As used herein, the term on-diagonal disulfide bonded cysteine pair refers to a pairing of a first cysteine residue to a second cysteine residue in a biologic, in a defined physical state, in relative high frequency compared to pairings between the same first cysteine residue and other cysteine residues, distinct from the second cysteine residue, and/or no cysteine residue in the same biologic, in the same predefined physical state. In some instances, an on-diagonal disulfide bonded cysteine pair is a disulfide-linked cysteine pair with an occupancy in a biologic of greater than 20%, 30%, 40%, 50%, 60%, 70%, 80%, 90%, 95%, or 99%.

As used herein, the term off-diagonal disulfide bonded cysteine pair refers to a pairing of a first cysteine residue to a second cysteine residue present in a biologic, in a defined physical state, in relative low frequency compared to pairings between the same first cysteine residue and other cysteine residues distinct from the second cysteine residue, and/or no cysteine residue in the same biologic, in the same defined physical state. In some instances, an on-diagonal disulfide bonded cysteine pair is a disulfide-linked cysteine pair with an occupancy in a biologic of less than 20%, 15%, 14%, 13%, 12%, 11%, 10%, 9%, 8%, 7%, 6%, 5%, 4%, 3%, 2%, or 1%.

As used herein, the term free cysteine refers to a cysteine residue present in a biologic, whether on-diagonal or off-diagonal, that is not involved in a disulfide bond.

As used herein, the term defined physical state refers to the arrangement of a biologic at a given time, as defined by the environment to which the biologic is exposed at the given time, wherein the environment is selected or controlled to essentially preserve disulfide bonding in the biologic. In some instances, the term predefined physical state refers to the arrangement of a biologic at a given time, as defined by the environment to which the biologic is exposed at the given time, wherein the environment is selected or controlled to essentially preserve disulfide bonds present in the biologic prior to analysis. In some instances, a given time is the time a disulfide bond profile is determined, e.g., when a biologic is analyzed to determine its disulfide bond profile.

In some instances, a disulfide bond profile can include quantitative information concerning the disulfide bond forming properties of one or more cysteines in the biologic. For example, such quantitative information can include the frequency, expressed as percent, in a biologic, in which a first cysteine associates with a second cysteine, relative to any other cysteine that the first cysteine can associate with (whether on-diagonal or off-diagonal). Such information is referred to herein as the occupancy of a cysteine residue (the first cysteine residue in a bonded pair). For example, assume that a given biologic has four cysteine residues, A, B, C, and D and that the first cysteine residue is A. If, in the biologic, 25% of A binds to B, 0% of A binds to C, and 75% of A binds to D, then the occupancy of A to D is 75%. Such quantitative information can also include the frequency, expressed as a percent, in a biologic, in which a disulfide bonded cysteine pair is present, relative to other disulfide bonded cysteine pairs. Such information is referred to herein as the abundance of a disulfide bonded cysteine pair. For example, in the given biologic, A-D has an abundance of 75%.

In some instances, a disulfide bond profile can include quantitative information concerning at least one on-diagonal disulfide bonded cysteine pair in the biologic.

In some instances, a disulfide bond profile can include quantitative information concerning at least two, three, four, five, six, or more, including all, on-diagonal disulfide bonded cysteine pairs in the biologic.

In some instances, a disulfide bond profile can include quantitative information concerning at least one, two, three, four, five, six, or more, including all, on-diagonal disulfide bonded cysteine pairs in the biologic, and quantitative and/or qualitative information concerning at least one, two, three, four, five, six, or more, including all, off-diagonal disulfide bonded cysteine pairs in the biologic.

In some instances, a disulfide bond profile is a disulfide bond profile shown in Table 3.

In some instances, a disulfide bond profile is a disulfide bond profile shown in Table 6.

In some instances, a disulfide bond profile is a disulfide bond profile shown in Table 9.

In some instances, information concerning relationships between cysteine residues present in a test biologic (e.g., a test protein), also referred to herein as a test protein disulfide bond profile conforms with (e.g., satisfies or meets, falls within (e.g., a range)) a disulfide bond profile (e.g., a disulfide bond profile for a target protein) if the test protein disulfide bond profile has a predetermined relationship the disulfide bond profile when the test and target are similarly processed (e.g., using the same method), wherein, when the predetermined relationship is identified, the test biologic qualifies as the target protein. In some instances, the predetermined relationship includes:

Conformity between one or more (e.g., 2, 3, 4, 5, 6, 7, 8, 9, 10, 11, 12, 13, 14, 15, or more than 15) on-diagonal parameters in the test protein with the equivalent or corresponding one or more (e.g., 2, 3, 4, 5, 6, 7, 8, 9, 10, 11, 12, 13, 14, 15, or more than 15) on-diagonal parameters in the disulfide bond profile for a target protein;

Conformity between one or more (e.g., 2, 3, 4, 5, 6, 7, 8, 9, 10, 11, 12, 13, 14, 15, or more than 15) off-diagonal parameters in the test protein with the equivalent or corresponding one or more (e.g., 2, 3, 4, 5, 6, 7, 8, 9, 10, 11, 12, 13, 14, 15, or more than 15) off-diagonal parameters in the disulfide bond profile for a target protein;

Conformity between one or more (e.g., 2, 3, 4, 5, 6, 7, 8, 9, 10, 11, 12, 13, 14, 15, or more than 15) on-diagonal parameters in the test protein with the equivalent or corresponding one or more (e.g., 2, 3, 4, 5, 6, 7, 8, 9, 10, 11, 12, 13, 14, 15, or more than 15) on-diagonal parameters in the disulfide bond profile for a target protein, and conformity between one or more (e.g., 2, 3, 4, 5, 6, 7, 8, 9, 10, 11, 12, 13, 14, 15, or more than 15) off-diagonal parameters in the test protein with the equivalent or corresponding one or more (e.g., 2, 3, 4, 5, 6, 7, 8, 9, 10, 11, 12, 13, 14, 15, or more than 15) off-diagonal parameters in the disulfide bond profile for a target protein; or

Conformity between information obtained for a test protein and all parameters in a disulfide bond profile.

In some instances, the test protein has a light chain amino acid sequence with at least 85%, 90%, 95%, 98%, 99%, or 100% identity to SEQ ID NO:1 and a heavy chain amino acid sequence with at least 85%, 90%, 95%, 98%, 99%, or 100% identity to SEQ ID NO: 2, and the predetermined relationship includes conformity between one or more parameters shown in Table 3 (where comparisons are made between the test protein disulfide bond profile and the corresponding between min-max values, “A” values, or “B” values), wherein the one or more parameters shown in Table 3 include:

Conformity between one or more (e.g., 2, 3, 4, 5, 6, 7, 8, 9, 10, 11, 12, 13, 14, 15, or more than 15) on-diagonal parameters in the test protein with the equivalent or corresponding one or more (e.g., 2, 3, 4, 5, 6, 7, 8, 9, 10, 11, 12, 13, 14, 15, or more than 15) on-diagonal parameters in the disulfide bond profile for a target protein;

Conformity between one or more (e.g., 2, 3, 4, 5, 6, 7, 8, 9, 10, 11, 12, 13, 14, 15, or more than 15) off-diagonal parameters in the test protein with the equivalent or corresponding one or more (e.g., 2, 3, 4, 5, 6, 7, 8, 9, 10, 11, 12, 13, 14, 15, or more than 15) off-diagonal parameters in the disulfide bond profile for a target protein;

Conformity between one or more (e.g., 2, 3, 4, 5, 6, 7, 8, 9, 10, 11, 12, 13, 14, 15, or more than 15) on-diagonal parameters in the test protein with the equivalent or corresponding one or more (e.g., 2, 3, 4, 5, 6, 7, 8, 9, 10, 11, 12, 13, 14, 15, or more than 15) on-diagonal parameters in the disulfide bond profile for a target protein, and conformity between one or more (e.g., 2, 3, 4, 5, 6, 7, 8, 9, 10, 11, 12, 13, 14, 15, or more than 15) off-diagonal parameters in the test protein with the equivalent or corresponding one or more (e.g., 2, 3, 4, 5, 6, 7, 8, 9, 10, 11, 12, 13, 14, 15, or more than 15) off-diagonal parameters in the disulfide bond profile for a target protein; or

Conformity between information obtained for a test protein and all parameters in a disulfide bond profile.

In some instances, the test protein has at least one (e.g., 2) amino acid sequence with at least 85%, 90%, 95%, 98%, 99%, or 100% identity to SEQ ID NO:3, and the predetermined relationship includes conformity between one or more parameters shown in Table 3 (where comparisons are made between the test protein disulfide bond profile and the corresponding between min-max values, “A” values, or “B” values), wherein the one or more parameters shown in Table 3 include:

Conformity between one or more (e.g., 2, 3, 4, 5, 6, 7, 8, 9, 10, 11, 12, 13, 14, 15, or more than 15) on-diagonal parameters in the test protein with the equivalent or corresponding one or more (e.g., 2, 3, 4, 5, 6, 7, 8, 9, 10, 11, 12, 13, 14, 15, or more than 15) on-diagonal parameters in the disulfide bond profile for a target protein;

Conformity between one or more (e.g., 2, 3, 4, 5, 6, 7, 8, 9, 10, 11, 12, 13, 14, 15, or more than 15) off-diagonal parameters in the test protein with the equivalent or corresponding one or more (e.g., 2, 3, 4, 5, 6, 7, 8, 9, 10, 11, 12, 13, 14, 15, or more than 15) off-diagonal parameters in the disulfide bond profile for a target protein;

Conformity between one or more (e.g., 2, 3, 4, 5, 6, 7, 8, 9, 10, 11, 12, 13, 14, 15, or more than 15) on-diagonal parameters in the test protein with the equivalent or corresponding one or more (e.g., 2, 3, 4, 5, 6, 7, 8, 9, 10, 11, 12, 13, 14, 15, or more than 15) on-diagonal parameters in the disulfide bond profile for a target protein, and conformity between one or more (e.g., 2, 3, 4, 5, 6, 7, 8, 9, 10, 11, 12, 13, 14, 15, or more than 15) off-diagonal parameters in the test protein with the equivalent or corresponding one or more (e.g., 2, 3, 4, 5, 6, 7, 8, 9, 10, 11, 12, 13, 14, 15, or more than 15) off-diagonal parameters in the disulfide bond profile for a target protein; or

Conformity between information obtained for a test protein and all parameters in a disulfide bond profile.

### Methods

In some instances, activities, actions, methods (such action steps are referred to collectively herein as “methods”) drawn to disulfide bond profiles in biologics include obtaining a sample of a batch of a test protein, optionally sequencing the test protein (e.g., using conventional sequencing techniques), determining a test protein disulfide bond profile for the sample, acquiring an assessment made by comparing the test protein disulfide bond profile determined for the sample with a disulfide bond profile for a target protein (e.g., a specification including a disulfide bond profile for a target protein), and conducting further activity when the comparison step yields or satisfies pre-determined information or criteria.

In some instances, methods drawn to disulfide bond profiles in biologics include determining (e.g., measuring or detecting) a test protein disulfide bond profile for a test biologic. Such determinations include identifying relationships between cysteine residues present in the biologic and can relate to on-diagonal and/or off-diagonal disulfide linked cysteines and/or free cysteine residues and can be qualitative and/or quantitative. In some instances, determining a test protein disulfide bond profile for a sample of a batch of a test protein includes, but is not limited to:

obtaining a sample of a batch of a test protein, and optionally obtaining, and optionally recording or memorializing (e.g., in paper or within a database) an amino acid sequence for the sample, wherein the amino acid sequence can represent the most abundant sequence for the biologic (e.g., the primary sequence), wherein the test protein is an antibody, e.g., a monoclonal antibody (e.g., a monoclonal antibody disclosed in Table 2, including, for example immunoglobulin isotype G (IgG), an IgG1 antibody, and IgG2 antibody, an antibody identified as a target protein in Table 2, or a biologic with a first amino acid sequence with at least 85%, 90%, 95%, 98%, 99%, or 100% identity to SEQ ID NO:1 and a second amino acid sequence with at least 85%, 90%, 95%, 98%, 99%, or 100% identity to SEQ ID NO:2; or an Fc fusion protein (e.g., a fusion protein disclosed in Table 2, including, for example, a CTLA4-Ig fusion protein, a biologic with an amino acid sequence with at least 85%, 90%, 95%, 98%, 99%, or 100% identity to SEQ ID NO:3);

processing the sample to obtain a material comprising a plurality of disulfide linked peptides, e.g., wherein each of the plurality of disulfide linked peptides includes no more than one, no more than two, no more than three, no more than four, or no more than five disulfide linked cysteine pairs;

analyzing the material comprising the plurality of disulfide linked peptides to obtain a test protein disulfide bond profile;

comparing information obtained for the test protein disulfide bond profile to corresponding information (e.g., a specification including a disulfide bond profile, parameters, and/or rules for a target protein) for a disulfide bond profile for a target protein, wherein the test protein and the target protein have at least a predefined amino acid sequence identity (e.g., wherein the test protein and the target protein have at least 85%, 90%, 95%, 98%, 99%, or 100% sequence identity, e.g., across their entire sequences, wherein the predefined amino acid sequence identity can be confirmed by comparing the amino acid sequence optionally obtained for the test protein with an amino acid sequence of the target protein); and

taking further action with respect to the test protein (e.g., confirming that the test protein qualifies as the target protein) if the test protein disulfide bond profile has a predetermined relationship with the disulfide bond profile.

In some instances, processing the sample to obtain a material comprising a plurality of disulfide linked peptides, disclosed above, includes cleaving (e.g., digesting) the sample to produce a plurality of disulfide linked peptides, e.g., wherein each of the plurality of disulfide linked peptides includes no more than one, no more than two, no more than three, no more than four, or no more than five disulfide linked cysteine pairs. In some instances, processing the sample to obtain a material comprising a plurality of disulfide linked peptides, disclosed above, includes treating (e.g., alkylating) the sample to block free cysteines present in the sample (e.g., to limit disulfide bond formation between free cysteines during subsequent cleavage), and cleaving (e.g., digesting) the sample to produce a plurality of disulfide bonded peptide fragments. In some instances, cleavage methods are selected to produce a plurality of disulfide linked peptides, e.g., wherein each of the plurality of disulfide linked peptides includes no more than one, no more than two, no more than three, no more than four, or no more than five disulfide linked cysteine pairs. In some instances, cleavage methods include enzymatic digestion selected to produce a plurality of disulfide linked peptides, e.g., wherein each of the plurality of disulfide linked peptides includes no more than one, no more than two, no more than three, no more than four, or no more than five disulfide linked cysteine pairs. In some instances, cleavage methods include enzymatic digestion selected to produce a plurality of disulfide linked peptides, e.g., wherein each of the plurality of disulfide linked peptides includes no more than one disulfide linked cysteine pairs. In some instances, cleavage methods include enzymatic digestion selected to remove glycans, e.g., glycans that interfere with production of disulfide linked peptides that include no more than one, no more than two, no more than three, no more than four, no more than five disulfide linked cysteine pairs. In some instances, cleavage methods include non-enzymatic methods to remove glycans, e.g., glycans that interfere with production of disulfide linked peptides that include no more than one, no more than two, no more than three, no more than four, no more than five disulfide linked cysteine pairs. In some instances, processing the sample to obtain a material comprising a plurality of disulfide linked peptides, disclosed above, includes selection and/or use of Method 1, Method 2, or Method 3, as exemplified herein, wherein:

In some instances, Method 1 is selected and/or used when the test biologic is an antibody, e.g., a monoclonal antibody (e.g., a monoclonal antibody disclosed in Table 2, including, for example, immunoglobulin isotype G (IgG), an IgG1 antibody, and IgG2 antibody, an antibody identified as a target protein in Table 2, or a biologic with a first amino acid sequence with at least 85%, 90%, 95%, 98%, 99%, or 100% identity to SEQ ID NO:1 and a second amino acid sequence with at least 85%, 90%, 95%, 98%, 99%, or 100% identity to SEQ ID NO:2); or

In some instances, Method 2 or Method 3 is selected when the biologic is a fusion protein, e g., an Fc fusion protein (e.g., a fusion protein disclosed in Table 2, including, for example, a CTLA4-Ig fusion protein, a biologic with an amino acid sequence with at least 85%, 90%, 95%, 98%, 99%, or 100% identity to SEQ ID NO:3).

In some instances, methods further include predicting disulfide linkages in a target protein, using those predicted disulfide linkages to obtain a disulfide bond profile for the target protein, and using the disulfide bond profile for the target protein in the methods disclosed herein. Such methods can include obtaining a forcibly scrambled form of the target biologic and using the forcibly scrambled form of the target biologic to identify on-diagonal and off-diagonal disulfide linkages in the target biologic. Resulting information is used to inform about the existence and/or prevalence of disulfide bonds in the target protein, including those that occur at relatively low levels, and thus may not otherwise have been detected.

As disclosed herein, obtaining a forcibly scrambled form of the target biologic includes disrupting native disulfide linkages in the biologic (including, e.g., disulfide linkages in all isoforms present in a biologic preparation), to a point that alternate disulfide bonds can subsequently reform. In some instances, disruption is accomplished using suitable chemical and/or physical methods. In some instances, disruption is accomplished using a denaturant (e.g., a chaeotropic agent) under conditions suitable to scramble disulfide bonds (e.g., at a temperature of about 37° C. (e.g., including about 20-40° C.), at about pH 8.0 (e.g., including about pH 6-10), for about 18 hours (e.g., including about 10-30 hours). Scrambling disulfide bonds are allowed to reform between free cysteines, yielding a forcibly scrambled form of the target biologic. The forcibly scrambled form of the sample is then processed and analyzed as disclosed herein (e.g., using Method 1, Method 2, or Method 3) and the resulting information is used to predict disulfide linkages in the target protein (e.g., to identify and quantify on-diagonal and/or off-diagonal disulfide linked cysteines and/or free cysteines) by informing about the existence and/or prevalence of disulfide bonds in the target protein.

In some instances, where a target protein is an antibody, e.g., a monoclonal antibody (e.g., a monoclonal antibody disclosed in Table 2, including, for example, immunoglobulin isotype G (IgG), an IgG1 antibody, and IgG2 antibody, an antibody identified as a target protein in Table 2, or a biologic with a first amino acid sequence with at least 85%, 90%, 95%, 98%, 99%, or 100% identity to SEQ ID NO:1 and a second amino acid sequence with at least 85%, 90%, 95%, 98%, 99%, or 100% identity to SEQ ID NO:2), predicting disulfide linkages in a target protein can include: identifying and quantifying on-diagonal disulfide linked cysteines using analytical methods disclosed herein; identifying at least one on-diagonal disulfide linked cysteine for an IgG antibody (e.g., an IgG1 or an IgG2 antibody) by reference to the literature (see, e.g., Huang et al., Analytical Chemistry, 84(11):4900 (2012) and/or Gall and Edelman, Biochemistry, 9(16):3188 (1970), each of which is hereby incorporated by reference in its entirety, or alternatively for its disclosure relating to disulfide linked cysteine pairs observed in IgG and associated methods) and quantifying the at least one on-diagonal disulfide linked cysteine identified by reference to the literature using methods disclosed herein.

In some instances, where a target protein is a fusion protein, e g., an Fc fusion protein (e.g., a fusion protein disclosed in Table 2, including, for example, a CTLA4-Ig fusion protein, a biologic with an amino acid sequence with at least 85%, 90%, 95%, 98%, 99%, or 100% identity to SEQ ID NO:3, predicting disulfide linkages in a target protein can include: identifying and quantifying on-diagonal disulfide linked cysteines using analytical methods disclosed herein.

In some instances, a biologic may undergo one or more steps prior to, subsequent to, or in addition to the methods described herein. For example, among other things, a biologic may be purified, fractionated, labeled, and/or digested.

As disclosed above, obtaining a forcibly scrambled form of the target biologic includes scrambling native disulfide linkages in the biologic (including, e.g., disulfide linkages in all isoforms present in a biologic preparation). Suitable methods are provided above. Other methods for obtaining a forcibly scrambled form of a biologic can include, for example, exposing a biologic to one or more reducing and/or denaturing agents that include, but are not limited to, dithiothreitol (DTT), 2-Mercaptoethanol (BME), 2-Mercaptoethylamine-HCl, Cysteine-HCl, TCEP-HCl, dihydrolipoic acid, and tris(2-carboxyethyl)phosphine), so long as such treatment is performed to a point that disulfide bonds can subsequently reform.

In some instances, free cysteines in a biologic can be blocked prior to or subsequent to cleavage (e.g., digestion). For example, a biologic preparation may be subjected to an alkylating agent. Suitable alkylating agents include, but are not limited to Iodoacetamide (IAM), d4-Iodoacetamide (d4-IAM), iodo acetic acid, N-ethylmaleamide (NEM), and 4-vinylpyridine (4VP), among others.

In instances that include a cleavage step, e.g., an enzyme digestion step, a biologic is exposed to one or more enzymes such as proteases or glycosidases (e.g., one, two, or three proteases and/or glycosidases). Suitable proteolytic enzymes include, for example, serine proteases, threonine proteases, cysteine proteases, aspartic acid proteases, metalloproteases, and glutamic acid proteases. Non-limiting examples of specific proteolytic enzymes that can be used in accordance with the present disclosure include trypsin, chymotrypsin, endoproteinase AspN, endoproteinase Lys C, elastase, subtilisin, proteinase K, pepsin, ficin, bromelin, plasmepsin, renin, chymosin, papain, a cathepsin (e.g., cathepsin K), a caspase (e.g., CASP3, CASP6, CASP7, CASP14), calpain 1, calpain 2, hermolysin, carboxypeptidase A or B, matrix metalloproteinase, a glutamic acid protease, and/or combinations thereof. Non-limiting examples of specific glycosidases that can be used in accordance with the present disclosure include β1-3 Galactosidase, β1-4 Galactosidase, β-N-Acetylglucosaminidase, α1-2,3 Mannosidase, α1-6 Mannosidase, α1-3,6 Galactosidase, α1-2 Fucosidase, PNGase F, Endoglycosidase F1, Endoglycosidase F2, and/or Endoglycosidase F3. Those of ordinary skill in the art will be aware of a number of other proteases or glycosidases that can be used in accordance with the present disclosure.

In some instances, a biologic is subjected to one or more enzymes (e.g., proteases and/or glycosidases) under conditions that minimize disruption of disulfide bonds. In some embodiments, cells are exposed to one or more protease enzymes for a limited period of time in order to avoid substantial disruption of disulfide bonds. For example, a biologic preparation may be subjected to one or more enzymes for a period of time that is less than about 15 minutes (e.g., less than about 14, 13, 12, 11, 10, 9, 8, 7, 6, 5, 4, 3, 2, or 1 minute(s)). In some embodiments, a biologic preparation is subjected to one or more enzymes for a period of time that is more than 15 minutes so long as substantial disruption of disulfide bonds does not occur. For example, a sufficiently low concentration of enzyme(s), a sufficiently low temperature and/or any of a variety of other factors or conditions may be employed such that the overall enzyme activity is decreased to a point where substantial disruption of disulfide bonds does not occur. Those of ordinary skill in the art will be aware of and will be able to employ factors or conditions that ensure that disruption of disulfide bonds does not occur.

In some instances, cleavage steps, e.g., enzymatic digestion steps, include use of/controlling/manipulating conditions that preserve disulfide connectivity during cleavage. Such methods can include, for example, use of pressure cycling technology. Exemplary conditions can include, one or more of: a temperature: of about 37° C. (e.g., 25-45° C.); high pressure: 20,000 PSI (e.g., 10,000-40,000 PSI); and time 1 (high pressure): 90 seconds (e.g., 30-360 seconds); time 2 (ambient pressure) 20 seconds (e.g., 1-100 seconds), with 35 cycles (e.g., with 1-100 cycles), and total digestion time of about 65 minutes (e.g., 1-120 minutes).

In some instances, cleavage can include, or can be substituted by, a non-enzymatic, e.g., chemical and/or physical treatment.

In one embodiment, analysis, determination, detection, and/or measuring, of a test protein disulfide bond profile and/or a disulfide bond profile for a target protein, includes use of any suitable mass spectrometry (MS) technique (e.g., ESI-MS, ESI-MS/MS, MALDI-TOF-MS, MALDI-TOF/TOF-MS, tandem MS, etc.). In some embodiments, a mass spectrometry technique can use electrospray ionization (ESI) to disperse liquid into a fine aerosol to generate ions. In ESI techniques, liquid containing analytes of interest typically include a volatile organic compound (e.g., methanol, acetonitrile, etc). During the ionization phase, the aerosol is sampled into a first vacuum stage through a capillary, where the solvent evaporates from a charged droplet until it becomes unstable, at which point the droplet deforms and loses a small percentage of its mass along with a relatively large percentage of its charge. Additional information relating to electrospray ionization is known to those of skill in the art.

In some embodiments, a mass spectrometry technique can use atmospheric pressure chemical ionization (APCI) in the positive ion mode to generate precursor positive ions. In APCI techniques, analytes of interest exist as charged species, such as protonated molecular ions [MH⁺] in the mobile phase. During the ionization phase, the molecular ions are desorbed into the gas phase at atmospheric pressure and then focused into the mass spectrometer for analysis and detection. Additional information relating to atmospheric pressure chemical ionization is known to those of skill in the art; see U.S. Pat. No. 6,692,971.

In some embodiments, selected reaction monitoring (SRM) may be used to analyze a biologic preparation. SRM is a non-scanning mass spectrometry technique, performed on triple quadrupole-like instruments and in which collision-induced dissociation is used as a means to increase selectivity. In SRM experiments, two mass analyzers are used as static mass filters, to monitor a particular fragment ion of a selected precursor ion. The specific pair of m/z values associated to the precursor and fragment ions selected is referred to as a “transition” and can be written as parent m/z >fragment m/z (e.g. 673.5>534.3). Unlike common MS based proteomics, no mass spectra are recorded in a SRM analysis. Instead, the detector acts as counting device for the ions matching the selected transition thereby returning an intensity value over time.

Multiple SRM transitions can be measured within the same experiment on the chromatographic time scale by rapidly toggling between the different precursor/fragment pairs (multiple reaction monitoring, MRM). In some embodiments, MRM may be used to analyze a biologic preparation. In MRM techniques, typically the triple quadrupole instrument cycles through a series of transitions and records the signal of each transition as a function of the elution time. MRM methods allow for additional selectivity by monitoring the chromatographic coelution of multiple transitions for a given analyte. In general, using MRM techniques, the specificity of precursor to product transitions may be harnessed for quantitative analysis of multiple proteins in a single sample. It will be appreciated that the design of MRM transitions is important for the success of MRM experiments.

MRM ion-pair transition data may be obtained and/or created by any available method, including methods known in the art. For example, MRM transition lists are publically and/or commercially available or may be custom-built. Software tools for creation of explicitly defined transition lists for MRM experiments are available, such as TPP-MARiMba, MRM Atlas Home, and MRMaid, Pinpoint, MIDAS (MRM Initiated Detection And Sequencing), and Skyline, among others.

In some embodiments, all cysteine-containing peptides and their theoretical masses may be tabulated. The m/z's of different charge states of all the possible disulfide pairs from these peptides may be calculated. The disulfide pairs that can be detected by this method may be established using the forced scrambled standard data based on full mass match (<5 ppm error) in combination with ms/ms fragmentation confirmation.

For relative quantification, each cysteine may be being considered individually. The relative abundance of each disulfide pair involving a particular cysteine may be normalized by all detectable disulfide bonds involving this cysteine. The relative quantitation of each disulfide pair may be described by the equations like below:

$$LC 23 - LC 88 disulfide \% = \frac{LC 23 - LC 88 disulfide}{\sum all LC 88 containing peptides}$$

$$LC 23 - LC 88 disulfide \% = \frac{LC 23 - LC 88 disulfide}{\sum all LC 23 containing peptides}$$

Since each disulfide may be quantified twice, one for each cysteine involved in the disulfide bond, an average of both values may be used to report the disulfide pair %.

The disulfide bond profile of the test protein may be compared to the disulfide bond profile of a target protein determined using the method described above.

As disclosed herein, methods include further activity with respect to the test protein when comparison step (iii) yields or satisfies pre-determined information or criteria. Such further activity can include, but is not limited to, for example, identifying, selecting, classifying, releasing, accepting, and/or categorizing, and/or using, a biologic, e.g., as suitable for or for commercial manufacture, use, sale, offer for sale, and/or importation, and/or discarding, withholding, processing (e.g., manufacturing) a drug substance into a drug product, processing (e.g., manufacturing) to drug substance, shipping, moving to a different location, formulating, labeling, packaging, when the preselected relationship is met. For example, a biologic (e.g., a test biologic) can be identified, classified, and/or categorized, e.g., as suitable for commercial manufacture, use, sale, offer for sale, and/or importation, by virtue of it having a defined or preselected disulfide bond profile. In some instances, such further activity can include converting a test protein to a pharmaceutical preparation or pharmaceutical composition, e.g., suitable for entry into commerce and/or administration to a subject (e.g., a human subject).

In some instances, methods (i.e., evaluation, identification, and production methods) can further include, e.g., one or more of: providing or obtaining a biologic preparation (e.g., such as a protein therapeutic or a precursor thereof); memorializing confirmation or identification of the biologic preparation using a recordable medium (e.g., on paper or in a computer readable medium, such as, in a Certificate of Testing, Certificate of Analysis, Material Safety Data Sheet (MSDS), batch record); informing a party or entity (e.g., contractual or manufacturing partner, a care giver or other end-user, a regulatory entity, such as, the FDA, or other U.S., European, Japanese, Chinese, or other governmental agency, or another entity, such as, a compendia entity (e.g., U.S. Pharmacopoeia (USP)), or insurance company) that a biologic preparation is a protein therapeutic; selecting the biologic preparation for further processing (e.g., processing, such as, formulating) the biologic preparation as a drug product (e.g., a pharmaceutical product) if the biologic preparation is identified as a protein therapeutic; and reprocessing or disposing of the biologic preparation if the biologic preparation is not identified as a protein therapeutic.

In some embodiments, provided methods may be combined with one or more other technologies for the detection, analysis, and/or isolation of polypeptides. It will be appreciated components of a biologic preparation may be separated according to methods known in the art prior to analysis.

In some instances, methods for evaluating a biologic preparation, e.g., the disulfide bond profile, in a biologic preparation are known in the art and/or are disclosed in Table 1:

TABLE 1
Exemplary methods of evaluating disulfide bond profiles

| Method(s) | Relevant literature | Parameter |
|---|---|---|
| Peptide LC-MS | Wang et al., Anal. Chem., | Free cysteine |
| (reducing/ | 83: 3133-3140 (2011); | |
| non-reducing) | Chumsae et al., Anal. Chem., | |
| | 81:6449-6457 (2009) | |
| Peptide LC-MS | Yan et al., J. Chrom. A., | Non-glycosylation-related peptide |
| (reducing/ | 1164: 153-161 (2007) | modifications (including, for example, |
| non-reducing) | Chelius et al., Anal. Chem., | sequence analysis and identification |
| | 78: 2370-2376 (2006) | of sequence variants; oxidation; |
| | Miller et al., J. Pharm. Sci., | succinimide; aspartic acid; and/or |
| | 100:2543-2550 (2011) | site-specific aspartic acid) |
| Weak cation | Dick et al., Biotechnol. Bioeng., | Isoforms (including, for example, |
| exchange (WCX) | 100: 1132-1143 (2008) | charge variants (acidic variants and |
| chromatography | | basic variants); and/or deamidated |
| | | variants) |
| Circular | Harn et al., Current Trends in | Secondary structure (including, for |
| dichroism | Monoclonal Antibody Development and | example, alpha helix content and/or |
| spectroscopy | Manufacturing, S. J. Shire et al., eds, | beta sheet content) |
| | 229-246 (2010) | |
| Intrinsic and/ | Harn et al., Current Trends in | Tertiary structure (including, for |
| or ANS dye | Monoclonal Antibody Development and | example, extent of protein folding) |
| fluorescence | Manufacturing, S. J. Shire et al., eds, | |
| | 229-246 (2010) | |

References listed in Table 1 are hereby incorporated by reference in their entirety, or in the alternative to the extent that they pertain to one or more of the methods disclosed in Table 1. Other methods for evaluating one or more parameters are disclosed elsewhere herein.

The provided methods achieve sample analysis that is one or more of: highly quantitative, high throughput, and useful to analyze small amounts of sample and/or low abundance elements (e.g., protein isoforms, free cysteines) present in a preparation. The provided methods also can be used to identify, classify, and/or categorize a biologic, e.g., as suitable for commercial manufacture, use, sale, offer for sale, and/or importation. For example, a biologic preparation can be identified, classified, and/or categorized, e.g., as suitable for commercial manufacture, use, sale, offer for sale, and/or importation, by virtue of having a defined or preselected disulfide bond profile.

While the present disclosure provides exemplary units and methods for the evaluation, identification, and production methods disclosed herein, a person of ordinary skill in the art will appreciate that performance of the evaluation, identification, and production methods herein is not limited to use of those units and/or methods. A person of skill in the art understands that although the use of other metrics or units (e.g., mass/mass, mole percent vs. weight percent) to measure a described parameter might give rise to different absolute values than those described herein, a test biologic meets a disclosed signature even if other units or metrics are used, as long as the test biologic meets the herein disclosed reference criterion or signature when the herein disclosed units and metrics are used, e.g., allowing for the sensitivity (e.g., analytical variability) of the method being used to measure the value.

### Target Biologics

As used herein, the terms “target biologic” or “target protein” refer to a commercially available, or approved, biologic which defines or provides the basis against which a test biologic is measured or evaluated. In some embodiments a target biologic is commercially available for therapeutic use in humans or animals. In other embodiments the target biologic was approved for use in humans or animals by a primary approval process. In other embodiments the target biologic is a reference listed drug for a secondary approval process. Examples of proteins that are target proteins in the United States include those in Table 1. In some instances, a target biologic is a monoclonal antibody that has a light chain amino acid sequence with at least 85% (e.g., at least 90%, 95%, 98%, 99%, or 100%) identity to SEQ ID NO:1 and a heavy chain amino acid sequence with at least 85% (e.g., at least 90%, 95%, 98%, 99%, or 100%) identity to SEQ ID NO:2. In some instances, a target biologic is a Fc-fusion protein with an amino acid sequence with at least 85% (e.g., at least 90%, 95%, 98%, 99%, or 100%) identity to SEQ ID NO:3. In other instances, a target biologic is protein or peptide with an amino acid sequence with at least 85% (e.g., at least 90%, 95%, 98%, 99%, or 100%) identity to the amino acid sequence of any one of the biologics listed in Table 2:

TABLE 2
Exemplary Target Biologics

| Protein Product | Reference Drug |
|---|---|
| raxibacumab | ABTHRAX ® |
| Tocilizumab | ACTERMRA ® |
| interferon gamma-1b | ACTIMMUNE ® |
| alteplase; tissue plasminogen | ACTIVASE ®/CATHFLO ® |
| activator | |
| brentuximab vedotin | ADCETRIS ® |
| Recombinant antihemophilic | ADVATE |
| factor | |
| human albumin | ALBUTEIN ® |
| Laronidase | ALDURAZYME ® |
| interferon alfa-N3, human | ALFERON N ® |
| leukocyte derived | |
| human antihemophilic factor | ALPHANATE ® |
| virus-filtered human | ALPHANINE ® SD |
| coagulation factor IX | |
| Alefacept; recombinant, dimeric | AMEVIVE ® |
| fusion protein LFA3-Ig | |
| Bivalirudin | ANGIOMAX ® |
| darbepoetin alfa | ARANESP ™ |
| rilonacept | ARCALYST ® |
| ofatumumab | ARZERRA ™ |
| Bevacizumab | AVASTIN ™ |
| interferon beta-1a; recombinant | AVONEX ® |
| coagulation factor IX | BENEFIX ™ |
| belimumab | BENLYSTA ® |
| Interferon beta-1b | BETASERON ® |
| Tositumomab | BEXXAR ® |
| antihemophilic factor | BIOCLATE ™ |
| human growth hormone | BIOTROPIN ™ |
| botulinum toxin type A | BOTOX ® |
| Alemtuzumab | CAMPATH ® |
| acritumomab; technetium-99 | CEA-SCAN ® |
| labeled | |
| alglucerase; modified form of | CEREDASE ® |
| beta-glucocerebrosidase | |
| imiglucerase; recombinant form | CEREZYME ® |
| of beta-glucocerebrosidase | |
| certolizumab pegol | CIMZIA ® |
| crotalidae polyvalent immune | CROFAB ™ |
| Fab, ovine | |
| digoxin immune Fab, ovine | DIGIFAB ™ |
| Rasburicase | ELITEK ® |
| Etanercept | ENBREL ® |
| epoietin alfa | EPOGEN ® |
| Cetuximab | ERBITUX ™ |
| arlibercept | EYLEA ® |
| algasidase beta | FABRAZYME ® |
| Urofollitropin | FERTINEX ™ |
| follitropin beta | FOLLISTIM ™ |
| Teriparatide | FORTEO ® |
| obinutuzumab | GAZYA ® |
| human somatropin | GENOTROPIN ® |
| Glucagon | GLUCAGEN ® |
| follitropin alfa | GONAL-F ® |
| antihemophilic factor | HELIXATE ® |
| Antihemophilic Factor; Factor XIII | HEMOFIL ® |
| Trastuzumab | HERCEPTIN ® |
| Insulin | HUMALOG ® |
| antihemophilic factor/von | HUMATE-P ® |
| Willebrand factor complex-human | |
| Somatotropin | HUMATROPE ® |
| Adalimumab | HUMIRA ™ |
| human insulin | HUMULIN ® |
| recombinant human hyaluronidase | HYLENEX ™ |
| canakinumab | ILARIS ® |
| interferon alfacon-1 | INFERGEN ® |
| Eptifibatide | INTEGRILIN ™ |
| alpha-interferon | INTRON A ® |
| trastuzumab emtansine | KADCYLA ® |
| Palifermin | KEPIVANCE |
| Anakinra | KINERET ™ |
| antihemophilic factor | KOGENATE ®FS |
| insulin glargine | LANTUS ® |
| granulocyte macrophage colony- | LEUKINE ®/LEUKINE ® LIQUID |
| stimulating factor | |
| ranibizumab | LUCENTIS ® |
| lutropin alfa, for injection | LUVERIS |
| OspA lipoprotein | LYMERIX ™ |
| Ranibizumab | LUCENTIS ® |
| gemtuzumab ozogamicin | MYLOTARG ™ |
| Galsulfase | NAGLAZYME ™ |
| Nesiritide | NATRECOR ® |
| Pegfilgrastim | NEULASTA ™ |
| Oprelvekin | NEUMEGA ® |
| Filgrastim | NEUPOGEN ® |
| Fanolesomab | NEUTROSPEC ™ |
| | (FORMERLY LEUTECH ®) |
| somatropin [rDNA] | NORDITROPIN ®/NORDITROPIN |
| | NORDIFLEX ® |
| insulin; zinc suspension; | NOVOLIN L ® |
| insulin; isophane suspension | NOVOLIN N ® |
| insulin, regular; | NOVOLIN R ® |
| Insulin | NOVOLIN ® |
| coagulation factor VIIa | NOVOSEVEN ® |
| romiplostim | NPLATE ® |
| belatacept | NULOJIX ® |
| Somatropin | NUTROPIN ® |
| immunoglobulin intravenous | OCTAGAM ® |
| PEG-L-asparaginase | ONCASPAR ® |
| denileukin diftitox | ONTAK ® |
| abatacept, fully human soluable | ORENCIA ™ |
| fusion protein | |
| muromomab-CD3 | ORTHOCLONE OKT3 ® |
| human chorionic gonadotropin | OVIDREL ® |
| peginterferon alfa-2a | PEGASYS ® |
| pegylated version of interferon | PEG-INTRON ™ |
| alfa-2b | |
| pertuzumab | PERJETA ® |
| Abarelix (injectable suspension); | PLENAXIS ™ |
| gonadotropin-releasing hormone | |
| antagonist | |
| epoietin alfa | PROCRIT ® |
| Aldesleukin | PROLEUKIN, IL-2 ® |
| denosumab | PROLIA ®/XGEVA ® |
| Somatrem | PROTROPIN ® |
| dornase alfa | PULMOZYME ® |
| Efalizumab; selective, | RAPTIVA ™ |
| reversible T-cell blocker | |
| combination of ribavirin and | REBETRON ™ |
| alpha interferon | |
| Interferon beta 1a | REBIF ® |
| antihemophilic factor | RECOMBINATE ® |
| rAHF/ntihemophilic factor | REFACTO ® |
| Lepirudin | REFLUDAN ® |
| Infliximab | REMICADE ® |
| Abciximab | REOPRO ™ |
| Reteplase | RETAVASE ™ |
| Rituximab | RITUXAN ™ |
| interferon alfa-2a | ROFERON-A ® |
| Somatropin | SAIZEN ® |
| synthetic porcine secretin | SECREFLO ™ |
| golimumab | SIMPONI ® |
| Basiliximab | SIMULECT ® |
| Eculizumab | SOLIRIS ® |
| Pegvisomant | SOMAVERT ® |
| ustekinumab | STELARA ® |
| Somatostatin | STILAMIN ® |
| Palivizumab; recombinantly | SYNAGIS ™ |
| produced, humanized mAb | |
| thyrotropin alfa | THYROGEN ® |
| Tenecteplase | TNKASE ™ |
| Natalizumab | TYSABRI ® |
| panitumumab | VECTIBIX ® |
| human immune globulin | VENOGLOBULIN-S ® |
| intravenous 5% and 10% | |
| solutions | |
| interferon alfa-n1, | WELLFERON ® |
| lymphoblastoid | |
| drotrecogin alfa | XIGRIS ™ |
| Omalizumab; recombinant DNA- | XOLAIR ® |
| derived humanized monoclonal | |
| antibody targeting | |
| immunoglobulin-E | |
| ipilimumab | YERVOY ® |
| Daclizumab | ZENAPAX ® |
| ibritumomab tiuxetan | ZEVALIN ™ |
| Somatotropin | ZORBTIVE ™ (SEROSTIM ®) |

In some instances, a target biologic is selected from the group consisting of: REMICADE®, RITUXAN®, PROLIA®/XGEVA®, AVASTIN®, HUMIRA®, HERCEPTIN®, TYSABRI®, STELARA®, SOLIRIS®, YERVOY®, XOLAIR®, ACTEMRA®, ERBITUX®, BENLYSTA®, SYNAGIS®, SIMPONI®, VECTIBIX®, ORENCIA®, ENBREL®, and EYLEA®.

### Test Biologics

As used herein, the terms test biologic or test protein refer to a commercially available biologic for therapeutic use in humans or animals that is not approved by a primary approval process. In some embodiments, the test biologic was approved for use in humans or animals by a secondary approval process. Methods for obtaining and/or manufacturing a test biologic for use in the applications disclosed herein are known in the art. Antibody biologic preparations can be generated using any available method, including methods well known in the art. For example, protocols for antibody production are described by Harlow and Lane, Antibodies: A Laboratory Manual, (1988). Typically, antibodies can be generated in rabbit, mouse, rat, guinea pig, hamster, camel, llama, shark, or other appropriate host. Alternatively, antibodies may be made in chickens, producing IgY molecules (Schade et al., (1996) ALTEX 13(5):80-85). In some embodiments, antibodies suitable for the present invention are subhuman primate antibodies. For example, general techniques for raising therapeutically useful antibodies in baboons may be found, for example, in Goldenberg et al., international patent publication No. WO 91/11465 (1991), and in Losman et al., Int. J. Cancer 46: 310 (1990). In some embodiments, monoclonal antibodies may be prepared using hybridoma methods (Milstein and Cuello, (1983) Nature 305(5934):537-40.). In some embodiments, monoclonal antibodies may be made by recombinant methods (U.S. Pat. No. 4,166,452, 1979).

In accordance with the present disclosure, there may be employed conventional molecular biology, microbiology, and recombinant DNA techniques within the skill of the art. Such techniques are described in the literature (see, e.g., Green & Sambrook, Molecular Cloning: A Laboratory Manual, Fourth Edition (2012) Cold Spring Harbor Laboratory Press, Cold Spring Harbor, N.Y.; DNA Cloning: A Practical Approach, Volumes I and II (Glover and Hames,eds. 1995); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); R. I. Freshney, Culture of Animal Cells: A Manual of Basic Technique and Specialized Application (2010); Immobilized Cells and Enzymes (IRL Press, (1986)); J. M. Guisan, Immobilization of Enzymes and Cells (2013); B. Perbal, A Practical Guide To Molecular Cloning (1984); T. A. Brown, Essential Molecular Biology: A Practical Approach Volume I (2000); T. A. Brown, Essential Molecular Biology: A Practical Approach Volume 11(2002); F. M. Ausubel et al. (eds.), Current Protocols in Molecular Biology, John Wiley & Sons, Inc. (1994).

### Applications

It will be appreciated that methods and techniques described herein can be utilized in any of a variety of applications. In general, these methods and techniques are useful in any application that involves the analysis of a biologic preparation that includes one or more disulfide bonds. One such application is in the manufacture of a therapeutic recombinant protein product. For example, information concerning the distribution of disulfide bonds within a biologic can be used to: identify the biologic as suitable for processing towards commercial release; for commercial release; compare target and test biologics, e.g., to determine the degree of similarity between the test and the target; and/or for monitoring change in a target or test biologic, such as a change that may result from the manufacture of the target or test biologic. In other words, provided techniques permit the identification, characterization, and/or quality control assessment of a biologic.

In some embodiments, any method described herein is performed using good manufacturing practices (GMP) as defined by the U.S. Food and Drug Administration (21 CFR Part 110).

Methods of the present disclosure can be utilized to analyze polypeptides and/or isoforms in any of a variety of states including, for instance, free polypeptides, or cells or cell components, etc.

The disclosure is further illustrated by the following examples. The examples are provided for illustrative purposes only. They are not to be construed as limiting the scope or content of the disclosure in any way

### EXAMPLES

### Example 1

### Identifying a Disulfide Bond Profile for Target Protein 1

Target Protein 1 is approved for use in the United States under a primary approval process (a biologics license application (BLA)) for various indications, including rheumatoid arthritis. Multiple batches of Target Protein 1 were analyzed using Method 1 to identify a disulfide bond profile for Target Protein 1. As disclosed herein, Method 1 is used to identify a disulfide bond profile for monoclonal antibodies.

### Method 1

Performance of Method 1 included, in summary: obtaining a sample of a batch of Target Protein 1 and processing the sample, including (i) an alkylation step, (ii) a buffer exchange step, and (iii) a cleavage step, including a step of digestion with a single enzyme. The resulting material was used to determine a disulfide bond profile using LC-MS/MS analysis. Method 1 included:

Obtaining a Sample of a Batch of Target Protein

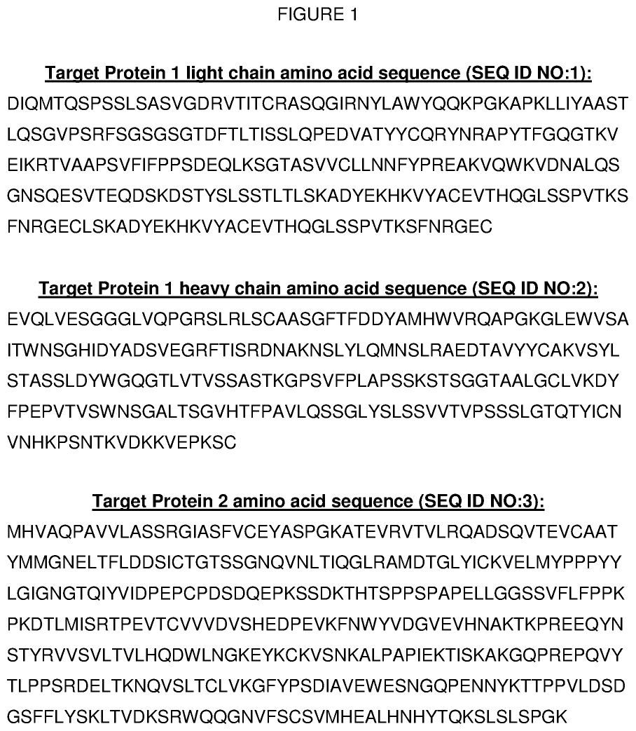

Samples of 13 batches of Target Protein 1 were obtained, and a portion of at least one of the samples was sequenced using conventional sequencing methods (sequencing of multiple samples/batches may be optionally performed). The light chain amino acid sequence of Target Protein 1 was determined and is shown as SEQ ID NO:1 (FIG. 1). The heavy chain amino acid sequence of Target Protein 1 was determined and is shown as SEQ ID NO:2. (FIG. 1).

### Sample Preparation

Alkylation: Prior to digestion, samples were treated with an alkylating agent under non-reducing conditions. Buffer Exchange: Resulting alkylated samples were buffer exchanged to mass spectrometry compatible pH 7.4. Digestion: Buffer exchanged samples were digested with a single enzyme (trypsin) using an enzyme:substrate ratio of 1:25. Digestion was performed in mass spectrometry compatible buffer using pressure cycling technology. Specifically, digestion was performed using a BAROCYCLER® NEP 2320 (Pressure Biosciences) with the Barocycler settings: temperature: 37° C.; high pressure: 20,000 PSI; time 1 (high pressure): 90 seconds; time 2 (ambient pressure) 20 seconds, with 35 cycles, and total digestion time of about 65 minutes. Digestion was quenched by adding formic acid to 2% (v/v).

### Determining a Disulfide Bond Profile

Processed samples were analyzed by C18 reversed phase and HPLC-MS peptide mapping run utilizing an Q Exactive Orbitrap mass spectrometer using suitable methods.

All cysteine-containing peptides and their theoretical masses were tabulated. The m/z's of different charge states of all the possible disulfide pairs from these peptides were calculated, as disclosed herein. Data for the thirteen batches analyzed is reported in Table 3.

TABLE 3
Disulfide bond profile of Target Protein 1

| | LC23 | | LC88 | | LC134 | | LC194 | | LC214 | | HC22 | | HC96 | | HC148 | |
|---|---|---|---|---|---|---|---|---|---|---|---|---|---|---|---|---|
| | MIN | MAX | MIN | MAX | MIN | MAX | MIN | MAX | MIN | MAX | MIN | MAX | MIN | MAX | MIN | MAX |
| LC23 | | | 99.77 | 99.99 | | | 0.00 | 0.01 | | | 0.00 | 0.18 | 0.00 | 0.14 | 0.00 | 0.02 |
| A | | | 94.78 | 104.99 | | | 0.00 | 0.01 | | | 0.00 | 0.19 | 0.00 | 0.15 | 0.00 | 0.02 |
| B | | | 79.82 | 119.99 | | | 0.00 | 0.01 | | | 0.00 | 0.22 | 0.00 | 0.17 | 0.00 | 0.02 |
| LC88 | 97.99 | 98.84 | | | | | | | | | 0.00 | 0.10 | 0.00 | 0.07 | | |
| A | 93.09 | 103.78 | | | | | | | | | 0.00 | 0.11 | 0.00 | 0.07 | | |
| B | 78.39 | 118.61 | | | | | | | | | 0.00 | 0.12 | 0.00 | 0.08 | | |
| LC134 | | | 0.00 | 0.01 | | | 99.41 | 99.69 | | | 0.02 | 0.27 | 0.00 | 0.25 | 0.00 | 0.01 |
| A | | | 0.00 | 0.01 | | | 94.44 | 104.67 | | | 0.02 | 0.28 | 0.00 | 0.26 | 0.00 | 0.01 |
| B | | | 0.00 | 0.01 | | | 79.53 | 119.63 | | | 0.02 | 0.32 | 0.00 | 0.30 | 0.00 | 0.01 |
| LC194 | 0.00 | 0.01 | | | 99.50 | 99.69 | | | | | 0.01 | 0.36 | 0.00 | 0.24 | 0.00 | 0.02 |
| A | 0.00 | 0.01 | | | 94.53 | 104.67 | | | | | 0.01 | 0.38 | 0.00 | 0.25 | 0.00 | 0.02 |
| B | 0.00 | 0.01 | | | 79.60 | 119.63 | | | | | 0.01 | 0.43 | 0.00 | 0.29 | 0.00 | 0.02 |
| LC214 | | | | | | | | | | | | | | | | |
| A | | | | | | | | | | | | | | | | |
| B | | | | | | | | | | | | | | | | |
| HC22 | 0.02 | 0.23 | 0.03 | 0.12 | 0.02 | 0.16 | 0.01 | 0.22 | | | | | 97.95 | 99.97 | 0.15 | 0.48 |
| A | 0.02 | 0.24 | 0.03 | 0.13 | 0.02 | 0.17 | 0.01 | 0.23 | | | | | 93.05 | 104.97 | 0.14 | 0.50 |
| B | 0.02 | 0.28 | 0.02 | 0.14 | 0.02 | 0.19 | 0.01 | 0.26 | | | | | 78.36 | 119.96 | 0.12 | 0.58 |
| HC96 | 0.00 | 0.17 | 0.00 | 0.08 | 0.00 | 0.14 | 0.00 | 0.14 | | | 94.54 | 98.06 | | | 0.00 | 0.30 |
| A | 0.00 | 0.18 | 0.00 | 0.08 | 0.00 | 0.15 | 0.00 | 0.15 | | | 89.81 | 102.96 | | | 0.00 | 0.32 |
| B | 0.00 | 0.20 | 0.00 | 0.10 | 0.00 | 0.17 | 0.00 | 0.17 | | | 75.63 | 117.67 | | | 0.00 | 0.36 |
| HC148 | 0.00 | 0.03 | | | 0.00 | 0.01 | 0.00 | 0.01 | 0.00 | 0.04 | 0.00 | 0.36 | 0.00 | 0.22 | | |
| A | 0.00 | 0.03 | | | 0.00 | 0.01 | 0.00 | 0.01 | 0.00 | 0.04 | 0.00 | 0.38 | 0.00 | 0.23 | | |
| B | 0.00 | 0.04 | | | 0.00 | 0.01 | 0.00 | 0.01 | 0.00 | 0.05 | 0.00 | 0.43 | 0.00 | 0.26 | | |
| HC204 | 0.00 | 0.09 | 0.00 | 0.02 | | | 0.00 | 0.06 | | | 0.00 | 0.36 | 0.00 | 0.79 | 99.20 | 99.88 |
| A | 0.00 | 0.09 | 0.00 | 0.02 | | | 0.00 | 0.06 | | | 0.00 | 0.38 | 0.00 | 0.83 | 94.24 | 104.87 |
| B | 0.00 | 0.11 | 0.00 | 0.02 | | | 0.00 | 0.07 | | | 0.00 | 0.43 | 0.00 | 0.95 | 79.36 | 119.86 |
| HC224 | | | | | | | | | 98.15 | 100.38 | | | | | | |
| A | | | | | | | | | 93.24 | 105.40 | | | | | | |
| B | | | | | | | | | 78.52 | 120.46 | | | | | | |
| HC230 | | | | | | | | | | | | | | | | |
| A | | | | | | | | | | | | | | | | |
| B | | | | | | | | | | | | | | | | |
| HC233 | | | | | | | | | | | | | | | | |
| A | | | | | | | | | | | | | | | | |
| B | | | | | | | | | | | | | | | | |
| HC265 | | | | | | | | | | | | | | | | |
| A | | | | | | | | | | | | | | | | |
| B | | | | | | | | | | | | | | | | |
| HC325 | | | | | | | | | | | | | | | | |
| A | | | | | | | | | | | | | | | | |
| B | | | | | | | | | | | | | | | | |
| HC371 | | | | | | | | | | | | | | | | |
| A | | | | | | | | | | | | | | | | |
| B | | | | | | | | | | | | | | | | |
| HC429 | | | | | | | | | | | | | | | | |
| A | | | | | | | | | | | | | | | | |
| B | | | | | | | | | | | | | | | | |
| Free/Alkylated | 0.82 | 1.95 | | | 0.17 | 0.35 | 0.20 | 0.31 | 0.00 | 1.86 | 0.00 | 4.68 | 0.23 | 0.55 | 0.00 | 14.57 |
| Cysteine | | | | | | | | | | | | | | | | |
| A | 0.78 | 2.05 | | | 0.16 | 0.37 | 0.19 | 0.33 | 0.00 | 1.95 | 0.00 | 4.91 | 0.22 | 0.58 | 0.00 | 15.30 |
| B | 0.66 | 2.34 | | | 0.14 | 0.42 | 0.16 | 0.37 | 0.00 | 2.23 | 0.00 | 5.62 | 0.18 | 0.66 | 0.00 | 17.48 |
| | HC204 | | HC224 | | HC230 | | HC233 | | HC265 | | HC325 | | HC371 | | HC429 | |
| | MIN | MAX | MIN | MAX | MIN | MAX | MIN | MAX | MIN | MAX | MIN | MAX | MIN | MAX | MIN | MAX |
| LC23 | 0.00 | 0.06 | | | | | | | | | | | | | | |
| A | 0.00 | 0.06 | | | | | | | | | | | | | | |
| B | 0.00 | 0.07 | | | | | | | | | | | | | | |
| LC88 | | | | | | | | | | | | | | | | |
| A | | | | | | | | | | | | | | | | |
| B | | | | | | | | | | | | | | | | |
| LC134 | | | | | | | | | | | | | | | | |
| A | | | | | | | | | | | | | | | | |
| B | | | | | | | | | | | | | | | | |
| LC194 | 0.00 | 0.19 | | | | | | | | | | | | | | |
| A | 0.00 | 0.20 | | | | | | | | | | | | | | |
| B | 0.00 | 0.23 | | | | | | | | | | | | | | |
| LC214 | | | 99.24 | 100.13 | | | | | | | | | | | | |
| A | | | 94.28 | 105.14 | | | | | | | | | | | | |
| B | | | 79.39 | 120.16 | | | | | | | | | | | | |
| HC22 | 0.04 | 0.31 | | | | | | | | | | | | | | |
| A | 0.04 | 0.33 | | | | | | | | | | | | | | |
| B | 0.03 | 0.37 | | | | | | | | | | | | | | |
| HC96 | 0.00 | 0.76 | | | | | | | | | | | | | | |
| A | 0.00 | 0.80 | | | | | | | | | | | | | | |
| B | 0.00 | 0.91 | | | | | | | | | | | | | | |
| HC148 | 97.29 | 97.93 | | | | | | | | | | | | | | |
| A | 92.43 | 102.83 | | | | | | | | | | | | | | |
| B | 77.83 | 117.52 | | | | | | | | | | | | | | |
| HC204 | | | | | | | | | | | | | | | | |
| A | | | | | | | | | | | | | | | | |
| B | | | | | | | | | | | | | | | | |
| HC224 | | | | | 0.00 | 1.04 | | | | | | | | | | |
| A | | | | | 0.00 | 1.09 | | | | | | | | | | |
| B | | | | | 0.00 | 1.25 | | | | | | | | | | |
| HC230 | | | 0.00 | 0.75 | 98.75 | 99.81 | | | | | | | | | | |
| A | | | 0.00 | 0.79 | 93.81 | 104.80 | | | | | | | | | | |
| B | | | 0.00 | 0.90 | 79.00 | 119.77 | | | | | | | | | | |
| HC233 | | | | | | | 98.93 | 100.01 | | | | | | | | |
| A | | | | | | | 93.98 | 105.01 | | | | | | | | |
| B | | | | | | | 79.14 | 120.01 | | | | | | | | |
| HC265 | | | | | | | | | 0.00 | 20.31 | 98.76 | 100.14 | | | | |
| A | | | | | | | | | 0.00 | 21.33 | 93.82 | 105.15 | | | | |
| B | | | | | | | | | 0.00 | 24.37 | 79.01 | 120.17 | | | | |
| HC325 | | | | | | | | | 93.57 | 97.93 | | | 0.00 | 0.02 | 0.00 | 0.02 |
| A | | | | | | | | | 88.89 | 102.83 | | | 0.00 | 0.02 | 0.00 | 0.02 |
| B | | | | | | | | | 74.86 | 117.52 | | | 0.00 | 0.02 | 0.00 | 0.02 |
| HC371 | | | | | | | | | | | 0.00 | 0.01 | | | 90.65 | 95.12 |
| A | | | | | | | | | | | 0.00 | 0.01 | | | 86.12 | 99.88 |
| B | | | | | | | | | | | 0.00 | 0.01 | | | 72.52 | 114.14 |
| HC429 | | | | | | | | | | | 0.00 | 0.02 | 93.85 | 96.11 | | |
| A | | | | | | | | | | | 0.00 | 0.02 | 89.16 | 100.92 | | |
| B | | | | | | | | | | | 0.00 | 0.02 | 75.08 | 115.33 | | |
| Free/Alkylated | 1.40 | 2.27 | | | 0.00 | 1.10 | 0.00 | 1.10 | 2.07 | 6.42 | 0.00 | 1.23 | 3.89 | 6.14 | 4.88 | 9.35 |
| Cysteine | | | | | | | | | | | | | | | | |
| A | 1.33 | 2.38 | | | 0.00 | 1.16 | 0.00 | 1.16 | 1.97 | 6.74 | 0.00 | 1.29 | 3.70 | 6.45 | 4.64 | 9.82 |
| B | 1.12 | 2.72 | | | 0.00 | 1.32 | 0.00 | 1.32 | 1.66 | 7.70 | 0.00 | 1.48 | 3.11 | 7.37 | 3.90 | 11.22 |

“LC” means ‘light chain’ and “HC” means ‘heavy chain’|Values represent min-max ranges of mean percent pairing for each parameter|“A” values represent 95%-105% of the min-max ranges of mean percent pairing for each parameter “B” values represent 80%-120% of the min-max ranges of mean percent pairing for each parameter.

The disulfide bond profile for Target Protein 1 shown in Table 3 is a signature of Target Protein 1 useful as a specification for determining that a test protein qualifies as Target Protein 1, as exemplified in Example 2.

### Example 2

### Processing Test Protein 1

The disulfide bond profile for Target Protein 1 was used to determine whether a batch of Test Protein 1 qualifies as Target Protein 1.

Test protein 1 is a monoclonal antibody against TNFα, representing a test biologic not approved under a primary approval process, that has a light chain with 100% identity to SEQ ID NO:1 and a heavy chain with 100% identity to SEQ ID NO: 2. A sample of a batch of Test Protein 1 was obtained and analyzed using Method 1, as disclosed in Example 1, and information was obtained for the parameters in Table 3. Resulting information is shown in Table 4:

TABLE 4
Disulfide bond profile for Test Protein 1

| | LC23 | LC88 | LC134 | LC194 | LC214 | HC22 | HC96 | HC148 | HC204 | HC224 |
|---|---|---|---|---|---|---|---|---|---|---|
| LC23 | | 99.99 | | | | | | | | |
| LC88 | 99.54 | | | | | | | | | |
| LC134 | | | | 99.79 | | 0.01 | | | | |
| LC194 | | | 99.76 | | | 0.01 | | | 0.01 | |
| LC214 | | | | | | | | | | 99.94 |
| HC22 | 0.01 | | 0.01 | 0.01 | | | 99.88 | 0.01 | 0.01 | |
| HC96 | | | | | | 99.52 | | 0.01 | | |
| HC148 | | | | | | 0.01 | | | 97.06 | |
| HC204 | | | | | | 0.01 | | 96.00 | | |
| HC224 | | | | | 99.96 | | | | | |
| HC230 | | | | | | | | | | 0.06 |
| HC233 | | | | | | | | | | |
| HC265 | | | | | | | | | | |
| HC325 | | | | | | | | | | |
| HC371 | | | | | | | | | | |
| HC429 | | | | | | | | | | |
| Cys | 0.45 | | 0.23 | 0.20 | 0.03 | 0.44 | 0.11 | 3.98 | 2.93 | |

| | HC230 | HC233 | HC265 | HC325 | HC371 | HC429 |
|---|---|---|---|---|---|---|
| LC23 | | | | | | |
| LC88 | | | | | | |
| LC134 | | | | | | |
| LC194 | | | | | | |
| LC214 | | | | | | |
| HC22 | | | | | | |
| HC96 | | | | | | |
| HC148 | | | | | | |
| HC204 | | | | | | |
| HC224 | 0.05 | | | | | |
| HC230 | 99.95 | | | | | |
| HC233 | | 100.00 | | | | |
| HC265 | | | | 99.43 | | |
| HC325 | | | 95.76 | | | |
| HC371 | | | | | | 92.37 |
| HC429 | | | | | 93.29 | |
| Cys | | | 4.42 | 0.57 | 6.70 | 7.63 |

“LC” means ‘light chain’ and “HC” means ‘heavy chain’|“Cys” means free and/or alkylated cysteine|Values represent percent pairing for each parameter

An assessment was acquired by comparing the information shown in Table 4 for Test Protein 1 with the “A” values for Target Protein 1 provided in Table 3. A summary of the assessment is shown in Table 5, wherein “✓” indicates compliance and “×” indicates non-compliance between the information shown in Table 4 and the “A” values in Table 3.

TABLE 5
Assessment of disulfide bond profile of Test Protein 1

| | LC23 | LC88 | LC134 | LC194 | LC214 | HC22 | HC96 | HC148 | HC204 | HC224 |
|---|---|---|---|---|---|---|---|---|---|---|
| LC23 | | ✓ | | | | | | | | |
| LC88 | ✓ | | | | | | | | | |
| LC134 | | | | ✓ | | x | | | | |
| LC194 | | | ✓ | | | x | | | ✓ | |
| LC214 | | | | | | | | | | ✓ |
| HC22 | x | | x | x | | | ✓ | x | x | |
| HC96 | | | | | | ✓ | | ✓ | | |
| HC148 | | | | | | | | | ✓ | |
| HC204 | | | | | | ✓ | | ✓ | | |
| HC224 | | | | | ✓ | | | | | |
| HC230 | | | | | | | | | | ✓ |
| HC233 | | | | | | | | | | |
| HC265 | | | | | | | | | | |
| HC325 | | | | | | | | | | |
| HC371 | | | | | | | | | | |
| HC429 | | | | | | | | | | |
| Cys | x | | ✓ | ✓ | ✓ | ✓ | x | x | x | |

| | HC230 | HC233 | HC265 | HC325 | HC371 | HC429 |
|---|---|---|---|---|---|---|
| LC23 | | | | | | |
| LC88 | | | | | | |
| LC134 | | | | | | |
| LC194 | | | | | | |
| LC214 | | | | | | |
| HC22 | | | | | | |
| HC96 | | | | | | |
| HC148 | | | | | | |
| HC204 | | | | | | |
| HC224 | ✓ | | | | | |
| HC230 | ✓ | | | | | |
| HC233 | | ✓ | | | | |
| HC265 | | | | ✓ | | |
| HC325 | | | ✓ | | | |
| HC371 | | | | | | ✓ |
| HC429 | | | | | ✓ | |
| Cys | | | ✓ | ✓ | x | ✓ |

“LC” means ‘light chain’ and “HC” means ‘heavy chain’|“✓” indicates compliance with a given rule

As shown in Table 5, the batch of Test Protein 1 would conform with the disulfide bond profile for Target Protein 1. Accordingly, Test Protein 1 would qualify as Target Protein 1.

### Example 3

### Identifying a Disulfide Bond Profile for Target Protein 2

Target protein 2 is a fusion protein approved for use in the United States under a primary approval process (a BLA) for various indications, including moderate to severe rheumatoid arthritis.

Three batches of Target Protein 2 were characterized using Method 2 to identify a disulfide bond profile for Target Protein 2. As disclosed herein, Method 2 is used to identify a disulfide bond profile for Fc fusion proteins.

### Method 2

Performance of Method 2 included, in summary: obtaining a sample of a batch of Target Protein 2 and processing the sample, including (i) an alkylation step, (ii) a buffer exchange step, and (iii) a two enzyme digestion step. The resulting sample was used to determine a disulfide bond profile using LC-MS/MS analysis. Method 2 included:

Obtaining a Sample of a Batch of Target Protein

Samples of batches of Target Protein 2 were obtained and a portion of at least one of the samples was sequenced using conventional sequencing methods. The amino acid sequence of Target Protein 2 is shown as SEQ ID NO:3 (FIG. 1).

Sample Preparation

Alkylation: Prior to digestion, samples were treated with an alkylating agent under non-reducing conditions. Buffer Exchange: Resulting alkylated samples were buffer exchanged into mass spectrometry compatible buffer at pH 7. Digestion: Buffer exchanged samples were digested using a two-enzyme cocktail of Glu C and trypsin with an enzyme:substrate of 1:20. Digestion was performed in the mass spectrometery compatible pH 7.0 using pressure cycling technology. Specifically, digestion was performed using a BAROCYCLER® NEP 2320 (Pressure Biosciences) with the Barocycler settings: temperature: 37° C.; high pressure: 20,000 PSI; time 1 (high pressure): 90 sec; time 2 (ambient pressure) 20 sec, with 35 cycles, and total digestion time of about 65 minutes. Digestion was quenched by adding formic acid to 2% (v/v).

Determining a Disulfide Bond Profile

Processed sample was analyzed by capillary C18 reversed phase and HPLC-MS peptide mapping run utilizing an Orbitrap XL mass spectrometer using suitable methods.

Values were calculated as disclosed in Example 1 for Target Protein 1. Data for the analyzed material is shown in Table 6.

TABLE 6
Disulfide bond profile of Target Protein 2

| | C21 | | C48 | | C66 | | C92 | | C120 | |
|---|---|---|---|---|---|---|---|---|---|---|
| | MIN | MAX | MIN | MAX | MIN | MAX | MIN | MAX | MIN | MAX |
| C21 | 0.00% | 1.41% | 0.75% | 2.78% | | | 99.58% | 99.62% | | |
| A | | 1.48% | 0.72% | 2.92% | | | 94.60% | 104.60% | | |
| B | | 1.69% | 0.60% | 3.34% | | | 79.66% | 119.54% | | |
| C48 | 0.00% | 0.05% | | | 100.00% | 100.00% | 0.00% | 0.04% | | |
| A | 0.00% | 0.05% | | | 95.00% | 105.00% | | | | |
| B | 0.00% | 0.06% | | | 80.00% | 120.00% | | | | |
| C66 | | | 83.48% | 90.70% | | | | | | |
| A | | | 79.30% | 95.24% | | | | | | |
| B | | | 66.78% | 108.84% | | | | | | |
| C92 | 98.25% | 98.57% | 1.06% | 2.37% | | | | | | |
| A | 93.34% | 103.50% | 1.01% | 2.49% | | | | | | |
| B | 78.60% | 118.28% | 0.85% | 2.84% | | | | | | |
| C120 | | | | | | | | | | |
| A | | | | | | | | | | |
| B | | | | | | | | | | |
| C171 | | | 0.61% | 0.61% | | | | | | |
| A | | | 0.58% | 0.64% | | | | | | |
| B | | | 0.49% | 0.73% | | | | | | |
| C231 | | | | | | | | | | |
| A | | | | | | | | | | |
| B | | | | | | | | | | |
| C277 | 0.07% | 0.09% | 1.58% | 2.45% | | | 0.07% | 0.09% | | |
| A | 0.06% | 0.09% | 1.50% | 2.57% | | | 0.06% | 0.09% | | |
| B | 0.05% | 0.11% | 1.27% | 2.94% | | | 0.05% | 0.11% | | |
| C335 | 0.00% | 0.04% | 0.30% | 2.86% | | | | | | |
| A | | | 0.29% | 3.00% | | | | | | |
| B | | | 0.24% | 3.43% | | | | | | |
| Free Cys | 0.22% | 1.13% | | | | | | | | |
| A | 0.21% | 1.19% | | | | | | | | |
| B | 0.18% | 1.36% | | | | | | | | |
| NEM | 0.20% | 0.21% | 3.97% | 5.58% | | | 0.26% | 0.29% | | |
| A | 0.19% | 0.22% | 3.77% | 5.86% | | | 0.24% | 0.30% | | |
| B | 0.16% | 0.25% | 3.17% | 6.70% | | | 0.20% | 0.35% | | |

| | C171 | | C231 | | C277 | | C335 | |
|---|---|---|---|---|---|---|---|---|
| | MIN | MAX | MIN | MAX | MIN | MAX | MIN | MAX |
| C21 | | | 0.86% | 0.86% | 0.07% | 0.10% | 0.00% | 0.05% |
| A | | | 0.81% | 0.90% | 0.07% | 0.11% | 0.00% | 0.05% |
| B | | | 0.69% | 1.03% | 0.06% | 0.12% | 0.00% | 0.06% |
| C48 | 0.48% | 1.27% | | | 0.00% | 0.05% | 0.00% | 0.06% |
| A | 0.46% | 1.33% | | | 0.00% | 0.05% | 0.00% | 0.06% |
| B | 0.39% | 1.52% | | | 0.00% | 0.06% | 0.00% | 0.07% |
| C66 | | | | | | | | |
| A | | | | | | | | |
| B | | | | | | | | |
| C92 | | | | | 0.07% | 0.10% | | |
| A | | | | | 0.07% | 0.11% | | |
| B | | | | | 0.06% | 0.12% | | |
| C120 | | | | | | | | |
| A | | | | | | | | |
| B | | | | | | | | |
| C171 | | | 99.14% | 100.00% | 0.07% | 0.09% | | |
| A | | | 94.19% | 105.00% | 0.07% | 0.09% | | |
| B | | | 79.31% | 120.00% | 0.06% | 0.11% | | |
| C231 | 40.15% | 48.27% | | | | | | |
| A | 38.15% | 50.68% | | | | | | |
| B | 32.12% | 57.92% | | | | | | |
| C277 | 8.38% | 11.12% | | | | | 89.02% | 90.87% |
| A | 7.96% | 11.68% | | | | | 84.57% | 95.41% |
| B | 6.70% | 13.34% | | | | | 71.21% | 109.04% |
| C335 | | | | | 80.53% | 83.13% | | |
| A | | | | | 76.51% | 87.29% | | |
| B | | | | | 64.43% | 99.76% | | |
| Free Cys | 7.98% | 9.46% | | | 0.68% | 0.89% | | |
| A | 7.58% | 9.93% | | | 0.64% | 0.93% | | |
| B | 6.39% | 11.35% | | | 0.54% | 1.07% | | |
| NEM | 35.31% | 40.13% | | | 15.88% | 18.29% | 8.94% | 10.92% |
| A | 33.54% | 42.14% | | | 15.09% | 19.20% | 8.49% | 11.47% |
| B | 28.25% | 48.16% | | | 12.70% | 21.95% | 7.15% | 13.10% |

“LC” means ‘light chain’ and “HC” means ‘heavy chain’|Values represent min-max ranges of mean percent pairing for each parameter|“A” values represent 95%-105% of the min-max ranges of mean percent pairing for each parameter|“B” values represent 80%-120% of the min-max ranges of mean percent pairing for each parameter.

The disulfide bond profile for Target Protein 2 shown in Table 6 refers to relationships between cysteine residues, parameters, including on-diagonal and/or off-diagonal disulfide linked cysteines and free cysteine residues, present in Target Protein 2. The disulfide bond profile for Target Protein 2 shown in Table 6 is a signature of Target Protein 2 useful as a specification for determining that a test protein qualifies as Target Protein 2, as exemplified in Example 4.

### Example 4

### Processing Test Protein 2

The disulfide bond profile for Target Protein 2 was used to determine whether a batch of Test Protein 2 qualifies as Target Protein 2.

Test Protein 2 is a fusion protein representing a test biologic not approved under a primary approval process, that has an amino acid sequence with 100% identity to SEQ ID NO:3. A sample of Test Protein 2 was obtained and analyzed using Method 2, as disclosed in Example 3, and information was obtained for the parameters in Table 6. Resulting information is shown in Table 7.

TABLE 7
Disulfide bond profile of Test Protein 2

| | C21 | C48 | C66 | C92 | C120 | C171 | C231 | C277 | C335 |
|---|---|---|---|---|---|---|---|---|---|
| C21 | 1.41 | 1.33 | | 99.62 | | | 0.86 | 0.08 | 0.05 |
| C48 | 0.02 | | 100.00 | 0.02 | | 0.48 | | 0.05 | 0.01 |
| C66 | | 89.80 | | | | | | | |
| C92 | 98.25 | 1.06 | | | | | | 0.07 | |
| C120 | | | | | | | | | |
| C171 | | | | | | | 99.14 | 0.08 | |
| C231 | 0.00 | | | | | 48.27 | | | |
| C277 | 0.07 | 2.45 | | 0.07 | | 11.12 | | | 89.02 |
| C335 | 0.04 | 0.30 | | | | | | 80.53 | |
| Free Cys | 0.22 | | | | | | | 0.89 | |
| Alkylated Cys | 0.20 | 4.88 | | 0.29 | | 40.13 | | 18.29 | 10.92 |

“LC” means ‘light chain’ and “HC” means ‘heavy chain’|“Cys” means free and/or alkylated cysteine|Values represent percent pairing for each parameter

An assessment was acquired by comparing the information shown in Table 7 for Test Protein 2 with the “A” values for Target Protein 2 provided in Table 6. A summary of the assessment is shown in Table 8, wherein “✓” indicates compliance and “×” indicates non-compliance between the information shown in Table 7 with the “A” values in Table 6.

TABLE 8
Assessment of disulfide bond profile of Test Protein 2

| | C21 | C48 | C66 | C92 | C120 | C171 | C231 | C277 | C335 |
|---|---|---|---|---|---|---|---|---|---|
| C21 | ✓ | ✓ | | ✓ | | | ✓ | ✓ | ✓ |
| C48 | ✓ | | ✓ | ✓ | | ✓ | | ✓ | ✓ |
| C66 | | ✓ | | | | | | | |
| C92 | ✓ | ✓ | | | | | | ✓ | |
| C120 | | | | | | | | | |
| C171 | | | | | | | ✓ | ✓ | |
| C231 | | | | | | ✓ | | | |
| C277 | ✓ | ✓ | | ✓ | | ✓ | | | ✓ |
| C335 | ✓ | ✓ | | | | | | ✓ | |
| Free | ✓ | | | | | | | ✓ | |
| Cys | | | | | | | | | |
| Alkylat- | ✓ | ✓ | | ✓ | | ✓ | | ✓ | ✓ |
| ed Cys | | | | | | | | | |

“LC” means ‘light chain’ and “HC” means ‘heavy chain’|“✓” indicates compliance with a given rule

As shown in Table 8, the batch of Test Protein 2 would conform with the disulfide bond profile for Target Protein 2. Accordingly, Test Protein 2 would qualify as Target Protein 2.

### Example 5

### Identifying a Disulfide Bond Profile for Target Protein 2

Target protein 2 is a fusion protein approved for use in the United States under a primary approval process (a BLA) for various indications, including moderate to severe rheumatoid arthritis.

Three batches of Target Protein 2 were characterized using Method 3 to identify a disulfide bond profile for Target Protein 2. As disclosed herein, Method 3 is used to identify a disulfide bond profile for Fc fusion proteins.

### Method 3

Performance of Method 3 included, in summary: obtaining a sample of a batch of Target Protein 2 and processing the sample, including (i) an alkylation step, (ii) a buffer exchange step, and (iii) a three enzyme digestion step. The resulting sample was used to determine a disulfide bond profile using LC-MS/MS analysis. Method 3 included:

Obtaining a Sample of a Batch of Target Protein

Samples of batches of Target Protein 2 were obtained and a portion of at least one of the samples was sequenced using conventional sequencing methods. The amino acid sequence of Target

Protein 2 is shown as SEQ ID NO:3 (FIG. 1).

Sample Preparation

Alkylation: Prior to digestion, samples were treated with an alkylating agent under non-reducing conditions. Buffer Exchange: Resulting alkylated samples were buffer exchanged into mass spectrometry compatible buffer at pH 7. Digestion: Buffer exchanged samples were digested using a three-enzyme cocktail of Glu C (Glu C:substrate—1:40 (w/w)), PNGaseF (PNGaseF:substrate—50 mU:1 mg), and trypsin (trypsin:substrate—1:20 (w/w)) with an enzyme:substrate of 1:20. Digestion was performed in the mass spectrometery compatible pH 7.0 using pressure cycling technology. Specifically, digestion was performed using a BAROCYCLER® NEP 2320 (Pressure Biosciences) with the Barocycler settings: temperature: 37° C.; high pressure: 20,000 PSI; time 1 (high pressure): 90 sec; time 2 (ambient pressure) 20 sec, with 35 cycles, and total digestion time of about 65 minutes. Digestion was quenched by adding formic acid to 2% (v/v).

Determining a Disulfide Bond Profile

Processed sample was analyzed by capillary C18 reversed phase and HPLC-MS peptide mapping run utilizing an Orbitrap XL mass spectrometer using suitable methods.

Values were calculated as disclosed in Example 1 for Target Protein 1. Data for the analyzed material is shown in Table 9.

TABLE 9
Disulfide bond profile of Target Protein 2

| | C21 | | C48 | | C66 | | C92 | | C120 | |
|---|---|---|---|---|---|---|---|---|---|---|
| | MIN | MAX | MIN | MAX | MIN | MAX | MIN | MAX | MIN | MAX |
| C21 | 0.01% | 0.02% | 0.04% | 0.04% | 0.05% | 0.10% | 99.32% | 99.58% | 0.42% | 0.63% |
| A | 0.01% | 0.02% | 0.04% | 0.04% | 0.05% | 0.11% | 94.35% | 104.56% | 0.40% | 0.66% |
| B | 0.01% | 0.02% | 0.03% | 0.05% | 0.04% | 0.12% | 79.46% | 119.50% | 0.34% | 0.76% |
| C48 | 0.08% | 0.11% | | | 99.09% | 99.41% | 0.04% | 0.06% | 0.65% | 1.23% |
| A | 0.08% | 0.12% | | | 94.14% | 104.38% | 0.04% | 0.06% | 0.62% | 1.29% |
| B | 0.06% | 0.13% | | | 79.27% | 119.29% | 0.03% | 0.07% | 0.52% | 1.48% |
| C66 | 0.09% | 0.19% | 99.85% | 99.87% | | | 0.12% | 0.33% | 1.05% | 1.26% |
| A | 0.09% | 0.20% | 94.86% | 104.86% | | | 0.11% | 0.35% | 1.00% | 1.32% |
| B | 0.07% | 0.23% | 79.88% | 119.84% | | | 0.10% | 0.40% | 0.84% | 1.51% |
| C92 | 99.31% | 99.50% | 0.02% | 0.02% | 0.05% | 0.16% | 0.01% | 0.02% | 0.39% | 1.14% |
| A | 94.34% | 104.48% | 0.02% | 0.02% | 0.05% | 0.17% | 0.01% | 0.02% | 0.37% | 1.20% |
| B | 79.45% | 119.40% | 0.02% | 0.02% | 0.04% | 0.19% | 0.01% | 0.02% | 0.31% | 1.37% |
| C120 | 0.01% | 0.01% | 0.01% | 0.01% | 0.01% | 0.01% | 0.01% | 0.02% | 86.74% | 89.96% |
| A | 0.01% | 0.01% | 0.01% | 0.01% | 0.01% | 0.01% | 0.01% | 0.02% | 82.40% | 94.46% |
| B | 0.01% | 0.01% | 0.01% | 0.01% | 0.01% | 0.01% | 0.01% | 0.02% | 69.39% | 107.95% |
| C171 | 0.01% | 0.01% | | | | | | | 1.75% | 3.75% |
| A | 0.01% | 0.01% | | | | | | | 1.66% | 3.94% |
| B | 0.01% | 0.01% | | | | | | | 1.40% | 4.50% |
| C231 | | | | | 0.01% | 0.01% | | | 1.04% | 2.89% |
| A | | | | | 0.01% | 0.01% | | | 0.99% | 3.03% |
| B | | | | | 0.01% | 0.01% | | | 0.83% | 3.47% |
| C277 | 0.07% | 0.08% | 0.02% | 0.02% | 0.03% | 0.04% | 0.06% | 0.07% | 0.68% | 0.87% |
| A | 0.07% | 0.08% | 0.02% | 0.02% | 0.03% | 0.04% | 0.06% | 0.07% | 0.65% | 0.91% |
| B | 0.06% | 0.10% | 0.02% | 0.02% | 0.02% | 0.05% | 0.05% | 0.08% | 0.54% | 1.04% |
| C335 | 0.10% | 0.11% | 0.03% | 0.03% | | | 0.06% | 0.09% | | |
| A | 0.10% | 0.12% | 0.03% | 0.03% | | | 0.06% | 0.09% | | |
| B | 0.08% | 0.13% | 0.02% | 0.04% | | | 0.05% | 0.11% | | |
| Free | 0.12% | 0.21% | 0.02% | 0.02% | 0.42% | 0.58% | 0.08% | 0.12% | 2.16% | 2.60% |
| Cys/NEM | | | | | | | | | | |
| A | 0.11% | 0.22% | 0.02% | 0.02% | 0.40% | 0.61% | 0.08% | 0.13% | 2.05% | 2.73% |
| B | 0.10% | 0.25% | 0.02% | 0.02% | 0.34% | 0.70% | 0.06% | 0.14% | 1.73% | 3.12% |

| | C171 | | C231 | | C277 | | C335 | |
|---|---|---|---|---|---|---|---|---|
| | MIN | MAX | MIN | MAX | MIN | MAX | MIN | MAX |
| C21 | 0.00% | 0.01% | | | 0.11% | 0.17% | 0.16% | 0.21% |
| A | | 0.01% | | | 0.10% | 0.18% | 0.15% | 0.22% |
| B | | 0.01% | | | 0.09% | 0.20% | 0.13% | 0.25% |
| C48 | | | | | 0.06% | 0.09% | 0.10% | 0.14% |
| A | | | | | 0.06% | 0.09% | 0.10% | 0.15% |
| B | | | | | 0.05% | 0.11% | 0.08% | 0.17% |
| C66 | 0.00% | 0.01% | 0.01% | 0.02% | 0.12% | 0.17% | | |
| A | | 0.01% | 0.01% | 0.02% | 0.11% | 0.18% | | |
| B | | 0.01% | 0.01% | 0.02% | 0.10% | 0.20% | | |
| C92 | | | | | 0.11% | 0.14% | 0.11% | 0.17% |
| A | | | | | 0.10% | 0.15% | 0.10% | 0.18% |
| B | | | | | 0.09% | 0.17% | 0.09% | 0.20% |
| C120 | 0.02% | 0.05% | 0.01% | 0.04% | 0.02% | 0.03% | | |
| A | 0.02% | 0.05% | 0.01% | 0.04% | 0.02% | 0.03% | | |
| B | 0.02% | 0.06% | 0.01% | 0.05% | 0.02% | 0.04% | | |
| C171 | | | 99.82% | 99.86% | 0.52% | 0.68% | 0.30% | 0.40% |
| A | | | 94.83% | 104.85% | 0.49% | 0.71% | 0.29% | 0.42% |
| B | | | 79.86% | 119.83% | 0.42% | 0.82% | 0.24% | 0.48% |
| C231 | 99.17% | 99.26% | | | 0.18% | 0.22% | 0.11% | 0.13% |
| A | 94.21% | 104.22% | | | 0.17% | 0.23% | 0.10% | 0.14% |
| B | 79.34% | 119.11% | | | 0.14% | 0.26% | 0.09% | 0.16% |
| C277 | 0.20% | 0.22% | 0.07% | 0.08% | 0.03% | 0.04% | 87.79% | 90.71% |
| A | 0.19% | 0.23% | 0.07% | 0.08% | 0.03% | 0.04% | 83.40% | 95.25% |
| B | 0.16% | 0.26% | 0.06% | 0.10% | 0.02% | 0.05% | 70.23% | 108.85% |
| C335 | 0.12% | 0.13% | 0.04% | 0.05% | 92.78% | 94.43% | 0.09% | 0.12% |
| A | 0.11% | 0.14% | 0.04% | 0.05% | 88.14% | 99.15% | 0.09% | 0.11% |
| B | 0.10% | 0.16% | 0.03% | 0.06% | 74.22% | 113.32% | 0.11% | 0.10% |
| Free | 0.37% | 0.41% | | | 4.40% | 5.69% | 8.41% | 11.09% |
| Cys/NEM | | | | | | | | |
| A | 0.35% | 0.43% | | | 4.18% | 5.97% | 7.99% | 11.64% |
| B | 0.30% | 0.49% | | | 3.52% | 6.83% | 6.73% | 13.31% |

“LC” means ‘light chain’ and “HC” means ‘heavy chain’|Values represent min-max ranges of mean percent pairing for each parameter|“A” values represent 95%-105% of the min-max ranges of mean percent pairing for each parameter|“B” values represent 80%-120% of the min-max ranges of mean percent pairing for each parameter.

The disulfide bond profile for Target Protein 2 shown in Table 9 refers to relationships between cysteine residues, parameters, including on-diagonal and/or off-diagonal disulfide linked cysteines and free cysteine residues, present in Target Protein 2. The disulfide bond profile for Target Protein 2 shown in Table 9 is a signature of Target Protein 2 useful as a specification for determining that a test protein qualifies as Target Protein 2, as exemplified in Example 6.

### Example 6

### Processing Test Protein 3

The disulfide bond profile for Target Protein 2 was used to determine whether a batch of Test Protein 3 qualifies as Target Protein 2.

Test Protein 3 is a fusion protein representing a test biologic not approved under a primary approval process, that has an amino acid sequence with 100% identity to SEQ ID NO:3. A sample of Test Protein 3 was obtained and analyzed using Method 3, as disclosed in Example 5, and information was obtained for the parameters in Table 9. Resulting information is shown in Table 10.

TABLE 10
Disulfide bond profile of Test Protein 3

| | C21 | C48 | C66 | C92 | C120 | C171 | C231 | C277 | C335 |
|---|---|---|---|---|---|---|---|---|---|
| C21 | 0.02 | 0.04 | 0.10 | 99.32 | 0.42 | 0.01 | 0.00 | 0.12 | 0.17 |
| C48 | 0.08 | 0.00 | 99.09 | 0.04 | 0.65 | 0.00 | 0.00 | 0.06 | 0.10 |
| C66 | 0.19 | 99.86 | 0.00 | 0.33 | 1.24 | 0.00 | 0.01 | 0.12 | 0.00 |
| C92 | 99.31 | 0.02 | 0.16 | 0.02 | 1.14 | 0.00 | 0.00 | 0.11 | 0.11 |
| C120 | 0.01 | 0.01 | 0.01 | 0.02 | 89.96 | 0.03 | 0.01 | 0.03 | 0.00 |
| C171 | 0.01 | 0.00 | 0.00 | 0.00 | 2.25 | 0.00 | 99.85 | 0.54 | 0.31 |
| C231 | 0.00 | 0.00 | 0.01 | 0.00 | 1.04 | 99.22 | 0.00 | 0.18 | 0.11 |
| C277 | 0.07 | 0.02 | 0.04 | 0.07 | 0.73 | 0.22 | 0.07 | 0.03 | 90.64 |
| C335 | 0.10 | 0.03 | 0.00 | 0.07 | 0.00 | 0.13 | 0.04 | 94.36 | 0.09 |
| Free | 0.21 | 0.02 | 0.58 | 0.12 | 2.58 | 0.39 | 0.00 | 4.44 | 8.46 |
| Cys/ | | | | | | | | | |
| Alkylat- | | | | | | | | | |
| ed Cys | | | | | | | | | |

“LC” means ‘light chain’ and “HC” means ‘heavy chain’|“Cys” means free and/or alkylated cysteine|Values represent percent pairing for each parameter

An assessment was acquired by comparing the information shown in Table 10 for Test Protein 3 with the “A” values for Target Protein 2 provided in Table 9. A summary of the assessment is shown in Table 11, wherein “✓” indicates compliance and “×” indicates non-compliance between the information shown in Table 10 with the “A” values in Table 9.

TABLE 11
Assessment of disulfide bond profile of Test Protein 2

| | C21 | C48 | C66 | C92 | C120 | C171 | C231 | C277 | C335 |
|---|---|---|---|---|---|---|---|---|---|
| C21 | ✓ | ✓ | ✓ | ✓ | ✓ | ✓ | | ✓ | ✓ |
| C48 | ✓ | | ✓ | ✓ | ✓ | | | ✓ | ✓ |
| C66 | ✓ | ✓ | | ✓ | ✓ | ✓ | ✓ | ✓ | |
| C92 | ✓ | ✓ | ✓ | ✓ | ✓ | | | ✓ | ✓ |
| C120 | ✓ | ✓ | ✓ | ✓ | ✓ | ✓ | ✓ | ✓ | |
| C171 | ✓ | | | | ✓ | | ✓ | ✓ | ✓ |
| C231 | | | ✓ | | ✓ | ✓ | | ✓ | ✓ |
| C277 | ✓ | ✓ | ✓ | ✓ | ✓ | ✓ | ✓ | ✓ | ✓ |
| C335 | ✓ | ✓ | | ✓ | | ✓ | ✓ | ✓ | ✓ |
| Free | ✓ | ✓ | ✓ | ✓ | ✓ | ✓ | | ✓ | ✓ |
| Cys/ | | | | | | | | | |
| Alkylat- | | | | | | | | | |
| ed Cys | | | | | | | | | |

“LC” means ‘light chain’ and “HC” means ‘heavy chain’|“✓” indicates compliance with a given rule

As shown in Table 11, the batch of Test Protein 3 would conform with the disulfide bond profile for Target Protein 2. Accordingly, Test Protein 3 would qualify as Target Protein 2.

### Other Embodiments

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

While the methods have been described in conjunction with various embodiments and examples, it is not intended that the methods be limited to such embodiments or examples. On the contrary, the present disclosure encompasses various alternatives, modifications, and equivalents, as will be appreciated by those of skill in the art.

While the methods have been particularly shown and described with reference to specific illustrative embodiments, it should be understood that various changes in form and detail may be made without departing from the spirit and scope of the present disclosure. Therefore, all embodiments that come within the scope and spirit of the present disclosure, and equivalents thereto, are intended to be claimed. The claims, descriptions and diagrams of the methods, systems, and assays of the present disclosure should not be read as limited to the described order of elements unless stated to that effect.

1. (canceled)

2. A method of manufacturing a pharmaceutical product, said method comprising:
obtaining a sample of a batch of a test biologic;
determining a disulfide bond profile for said sample;
acquiring an assessment made by comparing said determined disulfide bond profile with a disulfide bond profile of a target protein (e.g., a specification including a disulfide bond profile of a target protein), wherein said target protein is a biologic approved under a primary approval pathway;
processing said batch of said test biologic into a pharmaceutical product if said assessment reveals said determined disulfide bond profile conforms with said disulfide bond profile of said target protein;
thereby manufacturing a pharmaceutical product.

3. The method of claim 2, wherein said determining step comprises digesting said sample with one or more protease enzymes in a digestion buffer.

4. The method of claim 3, wherein said digestion buffer comprises trypsin, flavastacin, LysC, and/or GluC.

5. The method of claim 2, wherein said determining step comprises digesting said sample with no more than one protease enzyme in a digestion buffer.

6. The method of claim 5, wherein said digestion buffer comprises trypsin.

7. The method of claim 2, wherein said determining step comprises digesting said sample with at least two protease enzymes in a digestion buffer.

8. The method of claim 7, wherein said digestion buffer comprises no more than two protease enzymes.

9. The method of claim 8, wherein said digestion buffer comprises trypsin and GluC.

10. The method of claim 9, wherein said digestion buffer further comprises one or more glycosidase enzymes.

11. The method of claim 10, wherein said digestion buffer comprises trypsin, GluC, and PNGaseF.

12. The method of claim 11, wherein said test biologic is an antibody (e.g., a monoclonal antibody).

13. The method of claim 12, wherein said antibody is an IgG antibody.

14. The method of claim 13, wherein said IgG antibody is an IgG1 antibody.

15. The method of claim 12, wherein said antibody has a light chain with an amino acid sequence with at least 95% identity to SEQ ID NO:1 and a heavy chain with an amino acid sequence with at least 95% identity to SEQ ID NO:2.

16. The method of claim 2, wherein said test biologic is an antibody that has a light chain with an amino acid sequence with at least 95% identity to SEQ ID NO:1 and a heavy chain with an amino acid sequence with at least 95% identity to SEQ ID NO:2

17. The method of claim 15, wherein said antibody has a light chain with an amino acid sequence having 100% identity to SEQ ID NO:1 and a heavy chain with an amino acid sequence having 100% identity to SEQ ID NO:2.

18. The method of claim 2, wherein said test biologic is a fusion protein.

19. The method of claim 18, wherein said fusion protein is an Fc fusion protein.

20. The method of claim 18, wherein said fusion protein has an amino acid sequence having at least 95% identity to SEQ ID NO:3.

21. The method of claim 2, wherein said test biologic is a fusion protein having an amino acid sequence having at least 95% identity to SEQ ID NO:3.

22. The method of claim 20, wherein said fusion protein has an amino acid sequence having 100% identity to SEQ ID NO: 3.

23. A method of manufacturing a pharmaceutical product comprising an antibody having a light chain with an amino acid sequence having 100% identity to SEQ ID NO:1 and a heavy chain with an amino acid sequence having 100% identity to SEQ ID NO:2, said method comprising:
obtaining a sample of a batch of a test biologic, wherein said test biologic is an antibody having a light chain with an amino acid sequence having 100% identity to SEQ ID NO:1 and a heavy chain with an amino acid sequence having 100% identity to SEQ ID NO:2, and wherein said test biologic is approved under a secondary approval pathway;
determining a disulfide bond profile for said sample, wherein said determining comprises digesting said sample with no more than one protease enzyme in a digestion buffer;
acquiring an assessment made by comparing said determined disulfide bond profile with a disulfide bond profile of a target biologic, wherein said target biologic is an antibody having a light chain with an amino acid sequence having 100% identity to SEQ ID NO:1 and a heavy chain with an amino acid sequence having 100% identity to SEQ ID NO:2, wherein said target biologic is approved under a primary approval pathway;
processing said batch of said test biologic into a pharmaceutical product comprising an antibody having a light chain with an amino acid sequence having 100% identity to SEQ ID NO:1 and a heavy chain with an amino acid sequence having 100% identity to SEQ ID NO:2 if said assessment reveals said disulfide bond profile of said sample conforms with said disulfide bond profile of said target biologic;
thereby manufacturing a pharmaceutical product comprising an antibody having a light chain with an amino acid sequence having 100% identity to SEQ ID NO:1 and a heavy chain with an amino acid sequence having 100% identity to SEQ ID NO:2.

24. A method of manufacturing a pharmaceutical product comprising a fusion protein having an amino acid sequence having 100% identity to SEQ ID NO: 3, said method comprising:
obtaining a sample of a batch of test biologic, wherein said test biologic is a fusion protein having an amino acid sequence having 100% identity to SEQ ID NO: 3, and wherein said test biologic is approved under a secondary approval pathway;
determining a disulfide bond profile for said sample, wherein said determining comprises digesting said sample with no more than two protease enzymes in a digestion buffer;
acquiring an assessment made by comparing said test protein disulfide bond profile with a disulfide bond profile of a target biologic, wherein said target biologic is a fusion protein having an amino acid sequence having 100% identity to SEQ ID NO: 3, wherein said target biologic is approved under a primary approval pathway;
processing said batch of said test biologic into a pharmaceutical product comprising a fusion protein having an amino acid sequence having 100% identity to SEQ ID NO: 3 if said assessment reveals said disulfide bond profile of said sample conforms with said disulfide bond profile of said target biologic;
thereby manufacturing a pharmaceutical product comprising a fusion protein having an amino acid sequence having 100% identity to SEQ ID NO: 3.

25. A method of manufacturing a pharmaceutical product comprising a fusion protein having an amino acid sequence having 100% identity to SEQ ID NO: 3, said method comprising:
obtaining a sample of a batch of test biologic, wherein said test biologic is a fusion protein having an amino acid sequence having 100% identity to SEQ ID NO: 3, and wherein said test biologic is approved under a secondary approval pathway;
determining a disulfide bond profile for said sample, wherein said determining comprises digesting said sample with no more than two protease enzymes in a digestion buffer, wherein said digestion buffer further includes at least one glycosidase enzyme;
acquiring an assessment made by comparing said test protein disulfide bond profile with a disulfide bond profile of a target biologic, wherein said target biologic is a fusion protein having an amino acid sequence having 100% identity to SEQ ID NO: 3, wherein said target biologic is approved under a primary approval pathway;
processing said batch of said test biologic into a pharmaceutical product comprising a fusion protein having an amino acid sequence having 100% identity to SEQ ID NO: 3 if said assessment reveals said disulfide bond profile of said sample conforms with said disulfide bond profile of said target biologic;
thereby manufacturing a pharmaceutical product comprising a fusion protein having an amino acid sequence having 100% identity to SEQ ID NO: 3.
